(19) United States
(12) Patent Application Publication
(10) Pub. No.: US 2023/0228702 A1
(43) Pub. Date: Jul. 20, 2023

(54) GAS SENSOR

(71) Applicant: NGK Insulators, Ltd., Nagoya-shi (JP)

(72) Inventors: Yusuke WATANABE, Nagoya-shi (JP); Yasumasa FUJIOKA, Nagoya-shi (JP); Yohei GORO, Nagoya-shi (JP); Akihiro NAKAJIMA, Nagoya-shi (JP); Shotaro NIIZUMA, Nagoya-shi (JP)

(21) Appl. No.: 18/154,357

(22) Filed: Jan. 13, 2023

(30) Foreign Application Priority Data
Jan. 14, 2022 (JP) 2022-004224
Dec. 8, 2022 (JP) 2022-196270

(51) Int. Cl. G01N 27/409; G01N 27/41; G01N 33/00; G01N 27/406

(52) U.S. Cl. CPC G01N 27/409; G01N 27/41; G01N 33/0037; G01N 27/4067

(57) ABSTRACT

Provided is a gas sensor element capable of realizing highly accurate concentration measurement in both environments where the concentration of a specific gas in a measurement target gas is high and where the concentration is low. A gas sensor according to one aspect of the present invention determines whether the concentration of a predetermined gas component in a measurement target gas is higher or lower than a predetermined concentration. If it is determined that the concentration is lower, a specific temperature that a sensor element is to reach as a result of being heated by a heater unit is set to be lower than the specific temperature set if it is determined that the concentration is higher.

### CROSS-REFERENCE TO RELATED APPLICATION

The present application claims priority from Japanese application JP 2022-004224, filed on Jan. 14, 2022, and JP 2022-196270, filed on Dec. 8, 2022, the contents of which are hereby incorporated by reference into this application.

### TECHNICAL FIELD

The present invention relates to a gas sensor.

### BACKGROUND ART

Conventionally, some known gas sensors for detecting the concentration of a specific gas, such as oxygen or NOₓ, in a measurement target gas, such as exhaust gas from an automobile, have a sensor element with an internally embedded heater in order to activate an oxygen ion-conductive solid electrolyte that constitutes the sensor element. For example, JP 10-318979A discloses a gas sensor that has a sensor element with an internally embedded heater and that controls electric power of the heater such that the impedance of a measurement pump cell is constant.

JP 10-318979A is an example of related art.

### SUMMARY OF THE INVENTION

With the tightening of automobile emission regulations and the like, it is predicted that gas sensors will be required to be capable of not only highly accurate concentration measurement in environments where the concentration of a specific gas in the measurement target gas is high in addition to environments where the concentration of the specific gas is low. Studies from this perspective by the inventor of the present application lead to the invention finding that, with conventional gas sensors having a structure such as that described above, it is difficult to realize highly accurate concentration measurement in both environments where the concentration of the specific gas in the measurement target gas is high and highly accurate concentration measurement in environments where the concentration is low. This is described in detail below.

First, the inventor found that, in gas sensors that measure the concentration of the specific gas in the measurement target gas, generally, changes in the offset value significantly affect the measurement accuracy in environments where the concentration is low. That is, the concentration to be measured varies by several ppm depending on the change in the offset value. Therefore, even if the variation in the offset value is constant, the lower the concentration of the specific gas in the measurement target gas is, the more significantly a change in the offset value affects the measurement accuracy. For example, when the concentration of the specific gas in the measurement target gas is 500 ppm, an error caused by a change in the offset value remains at 1% even if the offset value changes by 5 ppm. In contrast, when the concentration of the specific gas in the measurement target gas is 50 ppm, the error caused by a change in the offset value is 10% if the offset value changes by 5 ppm, and the change in the offset value significantly affects the measurement accuracy.

Here, one possible cause of the change in the offset value is a change in the temperature of the sensor element (specifically, electrodes such as a measurement electrode) due to the set temperature of the heater, the temperature of the measurement target gas, or the like. For this reason, the inventor examined suppressing the change in the offset value by lowering the temperature of the sensor element, i.e., lowering the temperature of the heater.

As a result, the inventor found an issue where simply lowering the temperature of the sensor element (electrodes) may reduce the measurement accuracy. That is, lowering the temperature of the sensor element may suppress the decomposition reaction of the specific gas in the measurement target gas, thereby reducing the sensitivity to the specific gas. The measurement accuracy may deteriorate particularly when the concentration of the specific gas is high.

The present invention has been made in view of the foregoing situation in one aspect, and aims to provide a gas sensor element capable of realizing highly accurate concentration measurement in both environments where the concentration of the specific gas in the measurement target gas is high and environments where the concentration is low.

To solve the above-described problems, the present invention adopts the following configuration.

A gas sensor according to a first aspect includes: a sensor element formed by stacking a plurality of solid electrolyte layers having oxygen ion conductivity, the sensor element including: an internal cavity into which a measurement target gas is to be introduced; a measurement pump cell being an electrochemical pump cell including: a measurement electrode located in the internal cavity; an outer pump electrode located in a region different from the internal cavity; and a solid electrolyte layer, of the plurality of solid electrolyte layers, that is present between the measurement electrode and the outer pump electrode; and a heater unit embedded in the sensor element and configured to heat the sensor element to a specific temperature; a determination unit configured to determine, based on output of the measurement pump cell, whether a concentration of a predetermined gas component in the measurement target gas is higher or lower than a predetermined concentration; and a temperature setting unit configured to, if the determination unit determines that the concentration is lower, set the specific temperature to be lower than the specific temperature set if the determination unit determines that the concentration is higher.

In this configuration, if it is determined that the concentration of the predetermined gas component in the measurement target gas is low, the specific temperature that the sensor element is to reach as a result of being heated by the heater unit is set to be lower than the specific temperature set if it is determined that the concentration is high. In other words, the specific temperature is lowered when the concentration of the predetermined gas component is low, and the specific temperature is raised when the concentration of the predetermined gas component is high.

Thus, when the concentration of the predetermined gas component is low, the change in the offset value can be suppressed by lowering the specific temperature. Also, when the concentration of the predetermined gas component is high, the specific temperature can be raised to prevent the decomposition reaction of the specific gas in the measurement target gas from being suppressed. Also, the change in output in the case of using the gas sensor for a long period of time can also be suppressed by raising the specific temperature when the concentration of the predetermined gas component is high.

Accordingly, the gas sensor according to the first aspect can realize highly accurate concentration measurement in both environments where the concentration of the specific gas in the measurement target gas is high and where the concentration is low.

A gas sensor according to a second aspect may be the gas sensor according to the first aspect, wherein a slope of cell resistance of the measurement pump cell with respect to input power to the heater unit is 200 [ohm/W] or more. In this configuration, the slope of the cell resistance of the measurement pump cell with respect to the input power to the heater unit is 200 [ohm/W] or more. Here, if the value of the cell resistance of the measurement pump cell significantly changes in response to a change in the input power to the heater unit, the (change in) input power required to control the value of the cell resistance of the measurement pump cell can be reduced. That is, the value of the cell resistance of the measurement pump cell can be controlled with a small amount of (change in) input power to the heater unit by increasing the slope of the cell resistance of the measurement pump cell with respect to the input power to the heater unit. Further, the inventor confirmed through experiments that it is desirable that the slope of the cell resistance of the measurement pump cell with respect to the input power to the heater unit is, for example, 200 [ohm/W] or more in the atmosphere. Accordingly, the gas sensor according to the second aspect can control the value of the cell resistance of the measurement pump cell with a small amount of input power by setting the slope of the cell resistance of the measurement pump cell with respect to the input power to 200 [ohm/W] or more. Note that the slope of the cell resistance with respect to the input power to the heater unit refers to, for example, the slope of the cell resistance with respect to the input power to the heater unit in the atmosphere.

A gas sensor according to a third aspect may be the gas sensor according to the first or second aspect, wherein a slope of cell resistance of the measurement pump cell with respect to input power to the heater unit is 5000 [ohm/W] or less. In this configuration, the slope of the cell resistance of the measurement pump cell with respect to the input power to the heater unit is 5000 [ohm/W] or less. Here, as mentioned above, the value of the cell resistance of the measurement pump cell can be controlled with a small amount of (change in) input power to the heater unit by increasing the slope of the cell resistance of the measurement pump cell with respect to the input power to the heater unit. In addition, the inventor confirmed through experiments that it is desirable that the slope of the cell resistance of the measurement pump cell with respect to the input power to the heater unit is, for example, 5000 [ohm/W] or less in the atmosphere. Accordingly, the gas sensor according to the third aspect can control the value of the cell resistance of the measurement pump cell with a small amount of input power by setting the slope of the cell resistance of the measurement pump cell with respect to the input power to 5000 [ohm/W] or less.

A gas sensor according to a fourth aspect may be the gas sensor according to any one of the first to third aspects, wherein the sensor element further includes at least one adjustment pump cell, which is an electrochemical pump cell including: an inner pump electrode facing the internal cavity; the outer pump electrode, or a third electrode in contact with a solid electrolyte layer, of the plurality of solid electrolyte layers, and exposed to an external space; and a solid electrolyte layer, of the plurality of solid electrolyte layers, that is present between the inner pump electrode and the outer pump electrode or the third electrode, the measurement target gas from which oxygen contained therein has been pumped in the adjustment pump cell is introduced into the measurement pump cell, and a slope of cell resistance of the measurement pump cell with respect to input power to the heater unit is larger than a slope of cell resistance of the adjustment pump cell with respect to the input power to the heater unit.

In this configuration, the slope of the cell resistance of the measurement pump cell with respect to the input power to the heater unit is larger than the slope of the cell resistance of the adjustment pump cell with respect to the input power to the heater unit. That is, in this configuration, the slope of the cell resistance of the measurement pump cell with respect to the input power to the heater unit is large compared to the slope of the cell resistance of the adjustment pump cell with respect to the input power to the heater unit. Also, the slope of the cell resistance of the adjustment pump cell with respect to the input power to the heater unit is small compared to the slope of the cell resistance of the measurement pump cell with respect to the input power to the heater unit.

Adopting this configuration enables the gas sensor according to the fourth aspect to control the value of the cell resistance of the measurement pump cell with a small amount of input power, and does not make the temperature of the adjustment pump cell unnecessarily high or low.

As mentioned above, the value of the cell resistance of the measurement pump cell can be controlled with a small amount of (change in) input power to the heater unit by increasing the slope of the cell resistance of the measurement pump cell with respect to the input power to the heater unit.

Also, reducing the slope of the cell resistance of the adjustment pump cell with respect to the input power to the heater unit facilitates favorable control of the temperature of the adjustment pump cell, and enables control of the decomposition reaction of the predetermined gas in the measurement target gas.

That is, if the temperature of the adjustment pump cell is unnecessarily high, reaction between the predetermined gas in the measurement target gas and the inner pump electrode increases in the adjustment pump cell, resulting in excessively promoting the decomposition reaction of the predetermined gas in the measurement target gas. Further, if the temperature of the adjustment pump cell is unnecessarily low, the pump voltage at the adjustment pump cell increases, resulting in excessively promoting the decomposition reaction of the predetermined gas in the measurement target gas.

In contrast, in the gas sensor according to the fourth aspect, the slope of the cell resistance of the adjustment pump cell with respect to the input power to the heater unit is small, thus enabling favorable control of the temperature of the adjustment pump cell. Since the gas sensor according to the fourth aspect favorably controls the temperature of the adjustment pump cell, the temperature of the adjustment pump cell does not become unnecessarily high or low, and the decomposition reaction of the predetermined gas in the measurement target gas can be prevented from being excessively promoted.

Accordingly, the gas sensor according to the fourth aspect can control the value of the cell resistance of the measurement pump cell with a small amount of input power, and can favorably control the temperature of the adjustment pump cell to prevent the decomposition reaction of the predetermined gas from being excessively promoted.

A gas sensor according to a fifth aspect may be the gas sensor according to the fourth aspect, wherein, if the determination unit determines that the concentration is lower, a value of the cell resistance of the measurement pump cell is controlled so as to be a predetermined first value, and if the determination unit determines that the concentration is higher, the value of the cell resistance of the measurement pump cell is controlled so as to be a predetermined second value different from the first value. In this configuration, if it is determined that the concentration is low, the value of the cell resistance of the measurement pump cell is controlled so as to be the first value. If it is determined that the concentration is high, the value of the cell resistance of the measurement pump cell is controlled so as to be the second value. Therefore, the gas sensor according to the fifth aspect can prevent a situation where the measurement results change due to the passage of time (e.g., a change in the value of the cell resistance of the measurement pump cell) rather than the concentration of the predetermined gas component in the measurement target gas.

A gas sensor according to a sixth aspect may be the gas sensor according to the fourth or fifth aspect, wherein the slope of the cell resistance of the measurement pump cell with respect to the input power to the heater unit is 10 to 1000 times the slope of the cell resistance of the adjustment pump cell with respect to the input power to the heater unit. In this configuration, the slope of the cell resistance of the measurement pump cell with respect to the input power to the heater unit is 10 to 1000 times the slope of the cell resistance of the adjustment pump cell with respect to the input power to the heater unit. As mentioned above, it is desirable that the slope of the cell resistance of the measurement pump cell with respect to the input power to the heater unit is larger than the slope of the cell resistance of the adjustment pump cell with respect to the input power to the heater unit. The inventor confirmed through experiments that it is desirable to make the slope of the cell resistance of the measurement pump cell with respect to the input power to the heater unit 10 to 1000 times the slope of the cell resistance of the adjustment pump cell with respect to the input power to the heater unit. Accordingly, the gas sensor according to the sixth aspect can control the value of the cell resistance of the measurement pump cell with a small amount of input power, and can favorably control the temperature of the adjustment pump cell to prevent the decomposition reaction of the predetermined gas from being excessively promoted.

Note that as another mode of the gas sensor according to each of the above aspects, one aspect of the present invention may be an information processing method that realizes all or some of the above configurations, or may be a program, or may be a recording medium with such a program stored therein that can be read by a computer or any other device or machine. Here, the “recording medium that can be read by a computer or the like” refers to a medium in which information such as a program is stored by electrical, magnetic, optical, mechanical, or chemical action. The information processing method that realizes all or some of the above configurations may be referred to as, for example, a gas sensor control method or the like, as per the content of computations included. Similarly, the program that realizes all or some of the above configurations may be referred to as, for example, a gas sensor control program or the like.

For example, a gas sensor control method according to a seventh aspect is a method for controlling a gas sensor including a sensor element formed by stacking a plurality of solid electrolyte layers having oxygen ion conductivity, the sensor element including: an internal cavity into which a measurement target gas is to be introduced; a measurement pump cell being an electrochemical pump cell including: a measurement electrode located in the internal cavity; an outer pump electrode located in a region different from the internal cavity; and a solid electrolyte layer, of the plurality of solid electrolyte layers, that is present between the measurement electrode and the outer pump electrode; and a heater unit embedded in the sensor element and configured to heat the sensor element to a specific temperature, and is an information processing method for executing: a determination step of determining, based on output of the measurement pump cell, whether a concentration of a predetermined gas component in the measurement target gas is higher or lower than a predetermined concentration; and a temperature setting step of, if it is determined in the determination step that the concentration is lower, making the specific temperature lower than the specific temperature set if it is determined that the concentration is higher.

According to the present invention, a gas sensor element can be provided that is capable of realizing highly accurate concentration measurement in both environments where the concentration of the specific gas in the measurement target gas is high and environments where the concentration is low.

### BRIEF DESCRIPTION OF THE DRAWINGS

FIG. 1 schematically shows an example configuration of a gas sensor and includes a vertical cross-sectional view of a sensor element as viewed in the longitudinal direction thereof.

FIG. 2 shows an overview of sensor element drive temperature setting processing performed by the sensor shown in FIG. 1.

FIG. 3 shows an example regarding the conception of the slope of cell resistance of a measurement pump cell with respect to input power to a heater unit in the sensor shown in FIG. 1 and the like.

FIG. 4 shows an example regarding the conception of the slope of cell resistance of the measurement pump cell with respect to the input power to the heater unit in a sensor according to a variation and the like.

FIG. 5 shows an example of a functional configuration of a controller included in a sensor according to a variation.

### EMBODIMENTS OF THE INVENTION

An embodiment according to an aspect of the present invention (hereinafter also referred to as “this embodiment”) will be described with reference to the drawings. However, the following embodiment is merely an example of the present invention in all respects. It goes without saying that various improvements and variations can be made without departing from the scope of the invention. In other words, specific configurations suitable for an embodiment may be adopted as appropriate to carry out of the present invention.

A gas sensor according to this embodiment is a sensor that detects NOₓ using a sensor element having an internally embedded heater unit for heating the sensor element to a specific temperature, and measures the concentration of the detected NOR. The gas sensor according to this embodiment determines whether the NOₓ concentration is higher or lower than a reference concentration, based on the output of a measurement pump cell, which is an electrochemical pump cell included in the sensor element. If it is determined that the NOₓ concentration is lower than the reference concentration, the gas sensor according to this embodiment sets the specific temperature to be lower than the specific temperature set if it is determined that the NOₓ concentration is higher than the reference concentration.

Therefore, the gas sensor according to this embodiment can lower the specific temperature to prevent changes in an offset value when the NO concentration is low, and can increase the specific temperature to prevent the NO decomposition reaction from being suppressed when the NO concentration is high. An example of the gas sensor having the above configuration will be described below.

### Configuration Example

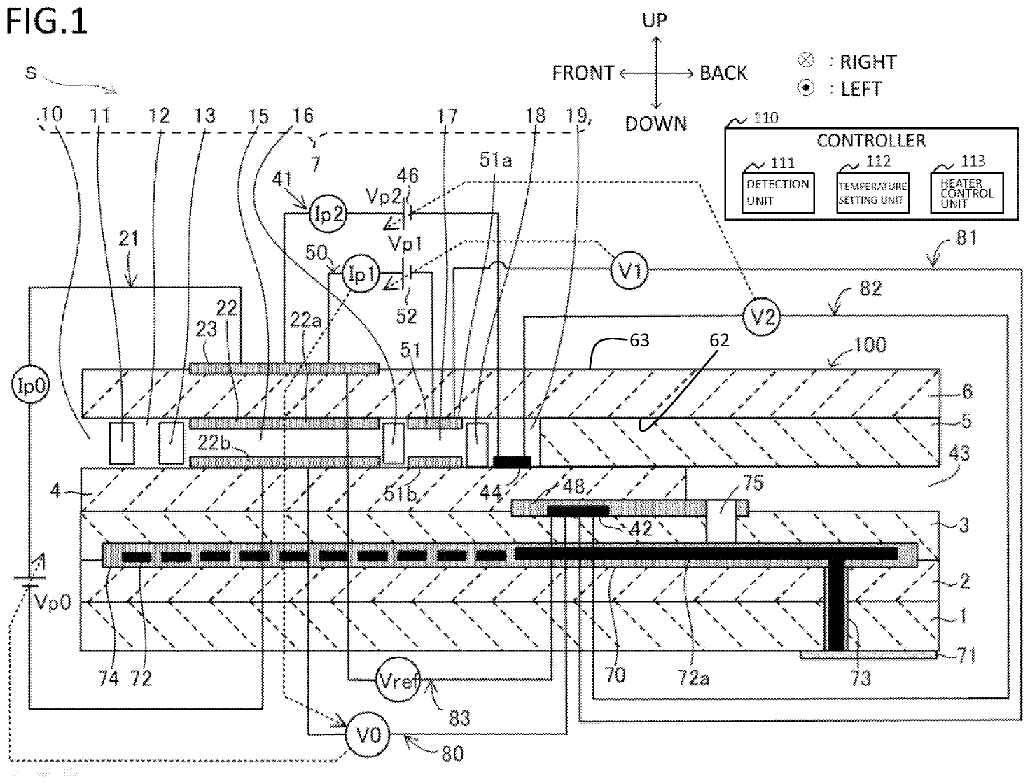

FIG. 1 schematically shows an example configuration of a gas sensor S and includes a vertical cross-sectional view of a gas sensor element 100 according to this embodiment as viewed in the longitudinal direction thereof. The gas sensor S includes the gas sensor element 100 and a controller 110, as shown in FIG. 1. The gas sensor element 100 has, for example, an elongated narrow plate body shape extending in the longitudinal direction (axial direction), and has, for example, a rectangular parallelepiped shape. The gas sensor element 100 illustrated in FIG. 1 has end portions in the longitudinal direction that are a leading end portion and a rear end portion. In the following description, the leading end portion corresponds to the left end portion in FIG. 1 (i.e., the end portion on the front side), and the rear end portion corresponds to the right end portion in FIG. 1 (i.e., the end portion on the rear side). However, the shape of the gas sensor element 100 need not be limited to this example, and may be selected as appropriate, as per the mode of implementation. Note that in the following description, the distal side of the paper plane of FIG. 1 corresponds to the right side of the gas sensor element 100, and the proximal side of the paper plane corresponds to the left side of the gas sensor element 100. The controller 110 has, as its functional elements, a determination unit 111, a temperature setting unit 112, and a heater control unit 113. The details of the gas sensor element 100 and the controller 110 will be described below.

### Gas Sensor Element

As illustrated in FIG. 1, the gas sensor element 100 includes a laminate constituted by a first substrate layer 1, a second substrate layer 2, a third substrate layer 3, a first solid electrolyte layer 4, a spacer layer 5, and a second solid electrolyte layer 6 that are stacked in this order from the lower side. These layers 1 to 6 are oxygen ion-conductive solid electrolyte layers made of zirconia (ZrO₂) or the like. The solid electrolyte that forms the layers 1 to 6 may be dense. Here, being dense means having a porosity of 5% or less.

The gas sensor element 100 is produced by, for example, performing steps such as predetermined processing and printing a wiring pattern on ceramic green sheets corresponding to respective layers, then stacking these layers, and firing and integrating them. As an example, the gas sensor element 100 is a laminate of a plurality of ceramic layers. In this embodiment, the upper face of the second solid electrolyte layer 6 constitutes the upper face of the gas sensor element 100, the lower face of the first substrate layer 1 constitutes the lower face of the gas sensor element 100, and side faces of the layers 1 to 6 constitute side faces of the gas sensor element 100.

In this embodiment, an internal space for receiving a measurement target gas from an external space is provided in one leading end portion of the gas sensor element 100 between a lower face 62 of the second solid electrolyte layer 6 and the upper face of the first solid electrolyte layer 4. The internal space according to this embodiment is formed such that a gas inlet 10, a first diffusion control portion 11, a buffer space 12, a second diffusion control portion 13, a first internal cavity 15, a third diffusion control portion 16, a second internal cavity 17, a fourth diffusion control portion 18, and a third internal cavity 19 are adjacent to each other in this order in a connected manner. In other words, the internal space according to this embodiment has a three-cavity structure (the first internal cavity 15, the second internal cavity 17, and the three internal cavity 19).

In one example, this internal space is formed by hollowing out a portion of the spacer layer 5. The upper portion of the internal space is demarcated by the lower face 62 of the second solid electrolyte layer 6. The lower portion of the internal space is demarcated by the upper face of the first solid electrolyte layer 4. The side portions of the internal space are demarcated by the side faces of the spacer layer 5.

The first diffusion control portion 11 is provided as two laterally elongated slits (the long sides of openings thereof extend in a direction perpendicular to the plane of FIG. 1). The second diffusion control portion 13, the third diffusion control portion 16, and the fourth diffusion control portion 18 are provided as holes whose length in a direction perpendicular to the plane of FIG. 1 are shorter than the first internal cavity 15, the second internal cavity 17, and the third internal cavity 19, respectively.

As illustrated in FIG. 1, the second diffusion control portion 13 and the third diffusion control portion 16 each may alternatively be provided as two laterally elongated slits (the long sides of openings thereof extend in a direction perpendicular to the plane of FIG. 1), similarly to the first diffusion control portion 11. On the other hand, the fourth diffusion control portion 18 may be provided as one laterally elongated slit (the long side of an opening thereof extends in a direction perpendicular to the plane of FIG. 1) that is formed as a gap below the lower face of the second solid electrolyte layer 6. That is, the fourth diffusion control portion 18 may be in contact with the upper face of the first solid electrolyte layer 4. The second diffusion control portion 13, the third diffusion control portion 16, and the fourth diffusion control portion 18 will be described later in detail. A region (internal space) from the gas inlet 10 to the third internal cavity 19 is referred to as a measurement target gas flow section 7.

A reference gas inlet space 43 having side portions demarcated by the side face of the first solid electrolyte layer 4 is provided between the upper face of the third substrate layer 3 and the lower face of the spacer layer 5, at a position farther from the leading end side (front side of the gas sensor element 100) than the measurement target gas flow section 7. A reference gas, such as the atmosphere, is introduced into the reference gas inlet space 43. However, the configuration of the gas sensor element 100 need not be limited to this example. As another example, the first solid electrolyte layer 4 may extend to the rear end of the gas sensor element 100, and the reference gas inlet space 43 may be omitted. In this case, an atmosphere inlet layer 48 may extend to the rear end of the gas sensor element 100.

The atmosphere inlet layer 48 is provided on a portion of the upper face of the third substrate layer 3 that is adjacent to the reference gas inlet space 43. The atmosphere inlet layer 48 is made of porous alumina, and the reference gas is introduced into the atmosphere inlet layer 48 via the reference gas inlet space 43. In addition, the atmosphere inlet layer 48 covers a reference electrode 42.

The reference electrode 42 is held between the first solid electrolyte layer 4 and the upper face of the third substrate layer 3, and is surrounded by the atmosphere inlet layer 48, which is connected to the reference gas inlet space 43. The reference electrode 42 is used to measure the oxygen concentration (oxygen partial pressure) in the first internal cavity 15 and the second internal cavity 17. The details will be described later.

The gas inlet 10 is a region of the measurement target gas flow section 7 that is open to the external space. The gas sensor element 100 is configured such that the measurement target gas is introduced from the external space to the inside through the gas inlet 10. In this embodiment, the gas inlet 10 is located in a leading end face (front face) of the gas sensor element 100, as illustrated in FIG. 1. In other words, the measurement target gas flow section 7 has an opening in the leading end face of the gas sensor element 100. However, it is not essential for the measurement target gas flow section 7 to have an opening in the leading end face of the gas sensor element 100, i.e., to dispose the gas inlet 10 in the leading end face of the gas sensor element 100 is not essential. The gas sensor element 100 need only be configured such that the measurement target gas can be introduced into the measurement target gas flow section 7 from the external space. For example, the gas inlet 10 may alternatively be located in the right face or left face of the gas sensor element 100.

The first diffusion control portion 11 is a region where predetermined diffusion resistance is applied to the measurement target gas introduced from the gas inlet 10.

The buffer space 12 is a space for guiding the measurement target gas introduced from the first diffusion control portion 11 to the second diffusion control portion 13.

The second diffusion control portion 13 is a region where predetermined diffusion resistance is applied to the measurement target gas introduced from the buffer space 12 to the first internal cavity 15.

When the measurement target gas is introduced into the first internal cavity 15 from the external space of the gas sensor element 100, there are cases where the measurement target gas is rapidly introduced into the gas sensor element 100 from the gas inlet 10 due to a change in the pressure of the measurement target gas in the external space (a pulsation of exhaust pressure in the case where the measurement target gas is exhaust gas from an automobile). Even in such cases, this configuration allows the introduced measurement target gas not to be introduced directly to the first internal cavity 15 but introduced to the first internal cavity 15 after a change in the concentration of the measurement target gas has been canceled out through the first diffusion control portion 11, the buffer space 12, and the second diffusion control portion 13. As a result, the change in the concentration of the measurement target gas introduced into the first internal cavity 15 is almost negligible.

The first internal cavity 15 is provided as a space for adjusting the oxygen partial pressure in the measurement target gas introduced through the second diffusion control portion 13. The oxygen partial pressure is adjusted by operation of the main pump cell 21.

The main pump cell 21 is an electrochemical pump cell that includes an inner pump electrode 22, an outer pump electrode 23, and the second solid electrolyte layer 6 held between these electrodes. The inner pump electrode 22 has a ceiling electrode portion 22a that is provided over the substantially entire surface of the lower face 62 of the second solid electrolyte layer 6 adjacent to (facing) the first internal cavity 15. The outer pump electrode 23 is provided in a region on an upper face 63 of the second solid electrolyte layer 6 that corresponds to the ceiling electrode portion 22a, and adjoins the external space. The main pump cell 21 is an example of an “adjustment pump cell”.

The inner pump electrode 22 is formed across the upper and lower solid electrolyte layers (the second solid electrolyte layer 6 and the first solid electrolyte layer 4) that demarcate the first internal cavity 15, and the spacer layer 5 that forms side walls of the first internal cavity 15. Specifically, the ceiling electrode portion 22a is formed on the lower face 62 of the second solid electrolyte layer 6 that forms a ceiling face of the first internal cavity 15, and a bottom electrode portion 22b is formed on the upper face of the first solid electrolyte layer 4 that forms a bottom face of the first internal cavity 15. Further, side electrode portions (not shown) that connect the ceiling electrode portion 22a to the bottom electrode portion 22b are formed on side wall faces (inner faces) of the spacer layer 5 that forms the two side wall portions of the first internal cavity 15. In other words, the inner pump electrode 22 is disposed in the form of a tunnel in the region where the side electrode portions are disposed. The inner pump electrode 22 is an example of an “inner pump electrode facing the internal cavity (measurement target gas flow section 7)”.

The inner pump electrode 22 and the outer pump electrode 23 are formed as porous cermet electrodes (e.g., cermet electrodes made of ZrO₂ and Pt containing 1% of Au). Note that the inner pump electrode 22, which comes into contact with the measurement target gas, is made of a material with a weakened capacity of reducing a nitrogen oxide (NOₓ) component in the measurement target gas.

The gas sensor element 100 is configured such that the main pump cell 21 applies a desired pump voltage Vp0 between the inner pump electrode 22 and the outer pump electrode 23 to cause a pump current Ip0 to flow in a positive or negative direction between the inner pump electrode 22 and the outer pump electrode 23, thereby enabling oxygen in the first internal cavity 15 to be pumped out to the external space, or oxygen in the external space to be pumped into the first internal cavity 15.

Furthermore, to detect the oxygen concentration (oxygen partial pressure) in the atmosphere in the first internal cavity 15, the inner pump electrode 22, the second solid electrolyte layer 6, the spacer layer 5, the first solid electrolyte layer 4, the third substrate layer 3, and the reference electrode 42 constitute an oxygen partial pressure detection sensor cell 80 for main pump control (i.e., an electrochemical sensor cell).

The gas sensor element 100 is capable of identifying the oxygen concentration (oxygen partial pressure) in the first internal cavity 15 by measuring an electromotive force V0 in the oxygen partial pressure detection sensor cell 80 for main pump control. Furthermore, the pump current Ip0 is controlled by performing feedback control on Vp0 so as to keep the electromotive force V0 constant. Accordingly, the oxygen concentration in the first internal cavity 15 can be kept at a predetermined constant value.

The third diffusion control portion 16 is a region where predetermined diffusion resistance is applied to the measurement target gas whose oxygen concentration (oxygen partial pressure) has been controlled in the first internal cavity 15 through the operation of the main pump cell 21, and the measurement target gas is guided to the second internal cavity 17.

The second internal cavity 17 is provided as a space for further adjusting the oxygen partial pressure in the measurement target gas introduced through the third diffusion control portion 16. The oxygen partial pressure is adjusted by operation of an auxiliary pump cell 50.

The auxiliary pump cell 50 is an auxiliary electrochemical pump cell constituted by an auxiliary pump electrode 51, the outer pump electrode 23 (not limited to the outer pump electrode 23 but need only be an appropriate electrode on the outer side of the gas sensor element 100), and the second solid electrolyte layer 6. The auxiliary pump electrode 51 has a ceiling electrode portion 51a located over a substantially entire portion facing the second internal cavity 17, of the lower face of the second solid electrolyte layer 6. The auxiliary pump cell 50 is an example of the “adjustment pump cell”. Also, the aforementioned “appropriate electrode on the outer side of the gas sensor element 100” is an example of a “third electrode in contact with the solid electrolyte layer and exposed to an external space”.

This auxiliary pump electrode 51 is disposed within the second internal cavity 17 in the form of a tunnel similar to the aforementioned inner pump electrode 22 provided within the first internal cavity 15. That is, the ceiling electrode portion 51a is formed on the lower face 62 of the second solid electrolyte layer 6 that forms the ceiling face of the second internal cavity 17, and a bottom electrode portion 51b is formed on the upper face of the first solid electrolyte layer 4 that forms the bottom face of the second internal cavity 17. Side electrode portions (not shown) that connect the ceiling electrode portion 51a to the bottom electrode portion 51b are formed on respective wall faces of the spacer layer 5 that form side walls of the second internal cavity 17. Thus, the auxiliary pump electrode 51 has a structure in the form of a tunnel. The auxiliary pump electrode 51 is an example of an “inner pump electrode facing an internal cavity (measurement target gas flow section 7)”.

Note that the auxiliary pump electrode 51 is also made of a material that has a weakened capacity of reducing a nitrogen oxide component in the measurement target gas, similarly to the internal pump electrode 22.

The gas sensor element 100 is configured such that the auxiliary pump cell 50 applies a desired voltage Vp1 between the auxiliary pump electrode 51 and the outer pump electrode 23, thereby enabling oxygen in the atmosphere in the second internal cavity 17 to be pumped out to the external space, or to be pumped into the second internal cavity 17 from the external space.

Furthermore, to control the oxygen partial pressure in the atmosphere in the second internal cavity 17, the auxiliary pump electrode 51, the reference electrode 42, the second solid electrolyte layer 6, the spacer layer 5, the first solid electrolyte layer 4, and the third substrate layer 3 constitute an oxygen partial pressure detection sensor cell 81 for auxiliary pump control (i.e., an electrochemical sensor cell).

Note that the auxiliary pump cell 50 performs pumping using a variable power source 52 whose voltage is controlled based on an electromotive force V1 detected by the oxygen partial pressure detection sensor cell 81 for auxiliary pump control. This controls the oxygen partial pressure in the atmosphere in the second internal cavity 17 to a partial pressure that is low enough to have substantially no impact on the NOₓ measurement.

Further, a pump current Ip1 is used together to control the electromotive force of the oxygen partial pressure detection sensor cell 80 for main pump control. Specifically, the pump current Ip1 is input as a control signal to the oxygen partial pressure detection sensor cell 80 for main pump control, and the electromotive force V0 is controlled so as to keep a constant gradient of the oxygen partial pressure in the measurement target gas introduced into the second internal cavity 17 from the third diffusion control portion 16. When this gas sensor is used as an NOₓ sensor, the oxygen concentration in the second internal cavity 17 is kept at a constant value of about 0.001 ppm by the operation of the main pump cell 21 and the auxiliary pump cell 50.

The fourth diffusion control portion 18 is a region where predetermined diffusion resistance is applied to the measurement target gas whose oxygen concentration (oxygen partial pressure) has been controlled by the operation of the auxiliary pump cell 50 in the second internal cavity 17, and this measurement target gas is guided to the third internal cavity 19.

The third internal cavity 19 is provided as a space for performing processing related to measurement of the nitrogen oxide (NOₓ) concentration in the measurement target gas introduced through the fourth diffusion control portion 18. The NOₓ concentration is measured by operation of a measurement pump cell 41. In this embodiment, the measurement target gas, which has been subjected to the pre-adjustment of the oxygen concentration (oxygen partial pressure) in the first internal cavity 15 and then introduced through the third diffusion control portion, is subjected to further adjustment of the oxygen partial pressure by the auxiliary pump cell 50 in the second internal cavity 17. This can accurately keep constant the oxygen concentration in the measurement target gas introduced into the third internal cavity 19 from the second internal cavity 17. Accordingly, the gas sensor element 100 according to this embodiment can accurately measure the NOₓ concentration.

The measurement pump cell 41 is used to measure the nitrogen oxide concentration in the measurement target gas in the third internal cavity 19. The measurement pump cell 41 is an electrochemical pump cell constituted by a measurement electrode 44, the outer pump electrode 23, the second solid electrolyte layer 6, the spacer layer 5, and the first solid electrolyte layer 4. In the example shown in FIG. 1, the measurement electrode 44 is located on the upper face of the first solid electrolyte layer 4 adjacent to (facing) the third internal cavity 19.

The measurement electrode 44 is a porous cermet electrode. The measurement electrode 44 also functions as an NOₓ reduction catalyst for reducing NOₓ that is present in the atmosphere in the third internal cavity 19. In the example shown in FIG. 1, the measurement electrode 44 is exposed in the third internal cavity 19. In another example, the measurement electrode 44 may be covered by a diffusion control portion. This diffusion control portion may be constituted by a porous film containing alumina (Al₂O₃) as a main component. The diffusion control portion serves to restrict the amount of NOₓ flowing into the measurement electrode 44, and also functions as a protective film for the measurement electrode 44.

The gas sensor element 100 is configured such that the measurement pump cell 41 can pump out oxygen generated through decomposition of nitrogen oxide in the atmosphere around the measurement electrode 44, and detect the amount of generated oxygen as a pump current Ip2.

To detect the oxygen partial pressure around the measurement electrode 44, the second solid electrolyte layer 6, the spacer layer 5, the first solid electrolyte layer 4, the third substrate layer 3, the measurement electrode 44, and the reference electrode 42 constitute an oxygen partial pressure detection sensor cell 82 for measurement pump control (i.e., an electrochemical sensor cell). A variable power source 46 is controlled based on a voltage (electromotive force) V2 detected by the oxygen partial pressure detection sensor cell 82 for measurement pump control.

The measurement target gas guided into the third internal cavity 19 reaches the measurement electrode 44 with the oxygen partial pressure controlled. Nitrogen oxide in the measurement target gas around the measurement electrode 44 is reduced to generate oxygen (2NO→N₂+O₂). The generated oxygen is pumped by the measurement pump cell 41. At that time, a voltage Vp2 of the variable power source is controlled such that the control voltage V2 detected by the oxygen partial pressure detection sensor cell 82 for measurement pump control is constant. The amount of oxygen generated around the measurement electrode 44 is proportional to the nitrogen oxide concentration in the measurement target gas. Therefore, the nitrogen oxide concentration in the measurement target gas is calculated using the pump current Ip2 in the measurement pump cell 41.

By combining the measurement electrode 44, the first solid electrolyte layer 4, the third substrate layer 3, and the reference electrode 42 to constitute an oxygen partial pressure detection means serving as an electrochemical sensor cell, it is possible to detect an electromotive force that corresponds to a difference between the amount of oxygen generated due to the reduction of an NOₓ component in the atmosphere around the measurement electrode 44 and the amount of oxygen contained in reference air. Thus, the concentration of the nitrogen oxide component in the measurement target gas can also be obtained.

Further, the second solid electrolyte layer 6, the spacer layer 5, the first solid electrolyte layer 4, the third substrate layer 3, the outer pump electrode 23, and the reference electrode 42 constitute an electrochemical sensor cell 83. The gas sensor element 100 is capable of detecting the oxygen partial pressure in the measurement target gas outside the sensor, based on an electromotive force Vref obtained by the sensor cell 83.

In the gas sensor element 100 having the above-described configuration, the measurement target gas whose oxygen partial pressure is always kept at a constant low value (a value that has no substantial impact on the NOₓ measurement) can be supplied to the measurement pump cell 41 by operating the main pump cell 21 and the auxiliary pump cell 50. Accordingly, the gas sensor element 100 is capable of identifying the nitrogen oxide concentration in the measurement target gas based on the pump current Ip2 that flows as a result of oxygen generated through the reduction of NOₓ being pumped out by the measurement pump cell 41, substantially in proportion to the nitrogen oxide concentration in the measurement target gas.

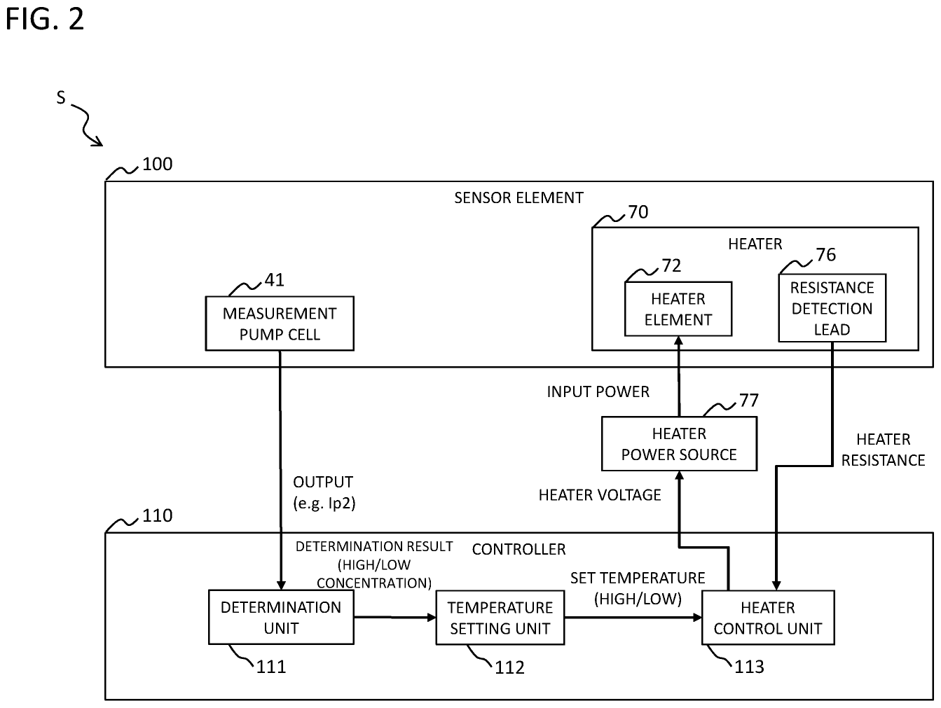

Furthermore, to improve the oxygen ion conductivity of the solid electrolyte, the gas sensor element 100 includes a heater 70 that serves to adjust the temperature by heating the gas sensor element 100 and retaining the temperature thereof. The heater 70 includes, as main components, heater electrodes 71 (for example, 71a, 71b, and 71c (not shown)), a heater element 72, heater leads 72a (for example, 72a1 and 72a2 (not shown)), through-holes 73, a heater insulating layer 74, and a heater resistance detection lead 76 (FIG. 2), which is not shown in FIG. 1. In the example in FIG. 1, the heater 70 also includes a pressure release hole 75.

The heater 70 is buried in a base portion of the gas sensor element 100, except for the heater electrodes 71. In this embodiment, the heater 70 is disposed at a position that is closer to the lower face of the gas sensor element 100 than to the upper face of the gas sensor element 100 in the thickness direction (vertical direction/stacking direction) of the gas sensor element 100. Note that the location of the heater 70 is not limited to this example, and may be selected as appropriate, as per the mode of implementation.

The heater electrodes 71 are electrodes formed in contact with the lower face of the first substrate layer 1 (the lower face of the gas sensor element 100). Electricity can be supplied from the outside to the heater 70 by connecting the heater electrodes 71 to an external power source.

The heater element 72 is an electrical resistor that is held from below and above by the second substrate layer 2 and the third substrate layer 3, i.e., heating resistors provided between the second substrate layer 2 and the third substrate layer 3. The heater element 72 is supplied electricity from a heater power source 77 (FIG. 2; not shown in FIG. 1) provided outside the gas sensor element 100 via an electricity flow path constituted by the heater electrodes 71, the through-holes 73, and the heater leads 72a, thereby generating heat to heat the solid electrolyte that forms the gas sensor element 100 and retain the temperature thereof. The heater element 72 is made of Pt, or contains Pt as a main component. The heater element 72 is buried in a predetermined area of the gas sensor element 100 on the side where the measurement target gas flow section 7 is located, and face the measurement target gas flow section 7 in the element thickness direction. Each heater element 72 has a thickness of about 10 μm to 20 μm, for example.

Two heater leads (for example, heater leads 72a1 and 72a2 (not shown)) connected to respective ends of each heater element 72 have substantially the same shape, i.e., the same resistance value. The heater leads 72a1 and 72a2 are connected respectively to different heater electrodes 71a and 71b (not shown) via corresponding through-holes 73.

Further, the heater resistance detection lead 76 is pulled out from a connecting portion between the heater element 72 and one of the heater leads, i.e., the heater lead 72a2. Note that the resistance value of the heater resistance detection lead 76 is negligible. The heater resistance detection lead 76 is connected to the heater electrode 71c (not shown) via a corresponding through-hole 73.

The heater element 72 is capable of adjusting the temperature of the entire gas sensor element 100 at a temperature that activates the solid electrolyte. That is, in the gas sensor element 100, each part of the gas sensor element 100 can be heated to a specific temperature and this temperature can be retained by causing a current to flow through the heater element 72 via the heater electrodes 71 to heat the heater element 72. Specifically, the gas sensor element 100 is heated such that the temperature of the solid electrolyte and the electrodes near the measurement target gas flow section 7 is about 700° C. to 900° C. (or 750° C. to 950° C.). This heating enhances the oxygen ion conductivity of the solid electrolyte constituting the base portion in the gas sensor element 100. Note that the heating temperature of the heater element 72 when the gas sensor S is used (i.e., when the gas sensor element 100 is driven) is referred to as a sensor element drive temperature in some cases.

The degree of heat generation by the heater element 72 is understood based on the magnitude of the resistance value (heater resistance) of the heater element 72. The heater resistance detection lead 76 is provided to measure the heater resistance.

The heater insulating layer 74 is an insulating layer formed so as to cover the heater element 72, e.g., an insulating layer that is formed on the upper and lower faces of the heater element 72 and made of an insulator such as alumina. The heater insulating layer 74 is formed for the purpose of achieving electrical insulation properties between the second substrate layer 2 and the heater element 72 and electrical insulation properties between the third substrate layer 3 and the heater element 72. The heater insulating layer 74 has a thickness of about 70 μm to 110 μm and is located at a position separated from the leading end face and side faces of the gas sensor element 100 by about 200 μm to 700 μm. Note that the thickness of the heater insulating layer 74 need not be constant, and may be different between a location where the heater element 72 is present and a location where the heater element 72 is not present.

The pressure release hole 75 is a region that passes through the third substrate layer 3 and is in communication with the reference gas inlet space 43. The pressure release hole 75 is formed for the purpose of mitigating the increase in the internal pressure due to a temperature rise in the heater insulating layer 74. Note that the provision of the pressure release hole 75 is not essential, and the pressure release hole 75 need not be provided.

### Controller

Next, the functions of the controller 110 will be described in detail. The controller 110 controls operation of each part of the gas sensor S, identifies the NOₓ concentration based on the pump current Ip2 flowing through the gas sensor element 100, and heats the gas sensor element 100 to a “specific temperature (sensor element drive temperature)” using the heater 70. The controller 110 is realized by a general-purpose or dedicated computer, and includes the determination unit 111, the temperature setting unit 112, and the heater control unit 113 as functional constituent elements realized by a CPU, memory, and the like of the computer, as illustrated in FIG. 1. Note that if the gas sensor S is for detecting and measuring NOₓ contained in exhaust gas from an automobile engine, and the gas sensor element 100 is attached to an exhaust path, some or all of the functions of the controller 110 may be realized by ECUs (electronic control units) installed in the automobile.

The functional blocks of the controller 110 in FIG. 1 and other diagrams include the determination unit 111, the temperature setting unit 112, and the heater control unit 113. However, the controller 110 may also include any functional block other than these functional blocks. For example, the controller 110 may include functional blocks for NOₓ detection, concentration calculation, or any other purposes. Specifically, the controller 110 may also include a functional block for controlling operation of each pump cell, a functional block for calculating NOₓ concentration, a functional block for comprehensively controlling operation of each part of the controller 110, or the like.

The determination unit 111 determines whether the NOₓ concentration in the measurement target gas is higher or lower than a predetermined concentration (reference concentration), based on the output of the measurement pump cell 41. For example, the determination unit 111 obtains the value of the pump current Ip2 flowing through the measurement pump cell 41, and determines whether the NOₓ concentration in the measurement target gas is higher or lower than the reference concentration, based on the obtained pump current Ip2. The determination unit 111 may determine whether the NOₓ concentration is higher or lower than the reference concentration by identifying the NOₓ concentration using a conventional method of identifying the NOₓ concentration in the measurement target gas based on the pump current Ip2 and comparing the identified NOₓ concentration with the reference concentration. The determination unit 111 may obtain the value of the pump current Ip2 flowing through the measurement pump cell 41, and identify the NOₓ concentration based on sensitivity characteristics data, which is a description of sensitivity characteristics that are preset for the gas sensor element 100. Such sensitivity characteristics may be identified in advance using a plurality of types of model gases with a known NOₓ concentration prior to actually using the gas sensor S, and sensitivity characteristics data, which is data regarding these sensitivity characteristics, may be stored in the controller 110.

The temperature setting unit 112 sets the specific temperature (sensor element drive temperature) that the gas sensor element 100 is to reach as a result of being heated by the heater 70. Specifically, if the determination unit 111 determines that the NOₓ concentration is lower than the reference concentration, the temperature setting unit 112 sets the specific temperature to be lower than the specific temperature set if the determination unit 111 determines that the NOₓ concentration is higher than the reference concentration. That is, the specific temperature that is set by the temperature setting unit 112 if the determination unit 111 determines that the NOₓ concentration is lower than the reference concentration is lower than the specific temperature that is set by the temperature setting unit 112 if the determination unit 111 determines that the NOₓ concentration is higher than the reference concentration.

The heater control unit 113 controls the operation of the heater 70. Specifically, the heater control unit 113 controls a heater voltage applied to the heater power source 77 such that the value of heater resistance (the resistance of the heater element 72), which is obtained as a resistance value between the heater resistance detection lead 76 and the heater lead 72a, is a value corresponding to the specific temperature set by the temperature setting unit 112. The heater control unit 113 thus controls the power supply to the heater 70, i.e., controls the input power to the heater 70. The heater element 72 generates an amount of heat corresponding to the heater resistance that is controlled in the above-described manner. When the heater control unit 113 controls the heater resistance value in accordance with the specific temperature set by the temperature setting unit 112, the gas sensor element 100 is heated by the heater 70 and reaches the specific temperature set by the temperature setting unit 112. The input power to the heater 70 refers to the product of the voltage applied to the heater 70 (i.e., the voltage applied across the heater) and the current flowing through the heater 70 (i.e., the current flowing in the heater).

### Sensor Element Drive Temperature Setting Processing

FIG. 2 shows an overview of sensor element drive temperature setting processing performed by the gas sensor S. In the sensor element drive temperature setting processing, first, the determination unit 111 obtains the output of the measurement pump cell 41 from the measurement pump cell 41, as illustrated in FIG. 2. For example, the determination unit 111 obtains, from the measurement pump cell 41, the value of the pump current Ip2 flowing through the measurement pump cell 41. The determination unit 111 determines whether the NOₓ concentration in the measurement target gas is higher or lower than the reference concentration, based on the obtained output (e.g., the pump current Ip2) of the measurement pump cell 41 (determination step). For example, the determination unit 111 determines whether the NOₓ concentration in the measurement target gas is higher or lower than the reference concentration by identifying the NOₓ concentration in the measurement target gas based on the pump current Ip2 and comparing the identified NOₓ concentration with the reference concentration. The determination unit 111 notifies the temperature setting unit 112 of the result (determination result) of the determination performed using the output of the measurement pump cell 41, i.e., whether the NOₓ concentration in the measurement target gas is higher or lower than the reference concentration.

The output of the measurement pump cell 41 that is used when the determination unit 111 determines whether the NOₓ concentration in the measurement target gas is higher or lower than the reference concentration is not limited to the value of the pump current Ip2. The determination unit 111 need only be capable of determining whether the NOₓ concentration in the measurement target gas is higher or lower than the reference concentration using any type of output of the measurement pump cell 41.

The temperature setting unit 112 sets the specific temperature that the gas sensor element 100 is to reach as a result of being heated by the heater 70, i.e., the sensor element drive temperature, based on the determination result that the temperature setting unit 112 has been notified of by the determination unit 111. Here, if the determination unit 111 determines that the NOₓ concentration is lower than the reference concentration, the temperature setting unit 112 sets the sensor element drive temperature to be lower than the sensor element drive temperature set if the determination unit 111 determines that the NOₓ concentration is higher than the reference concentration (temperature setting step).

That is, if the determination unit 111 determines that the NOₓ concentration is lower than the reference concentration, the temperature setting unit 112 sets a lower sensor element drive temperature than the sensor element drive temperature that is set if the determination unit 111 determines that the NOₓ concentration is higher than the reference concentration. Also, if the determination unit 111 determines that the NOₓ concentration is higher than the reference concentration, the temperature setting unit 112 sets a higher sensor element drive temperature than the sensor element drive temperature that is set if the determination unit 111 determines that the NOₓ concentration is lower than the reference concentration. Note that, if the determination unit 111 determines that the NOₓ concentration is equal to the reference concentration, the temperature setting unit 112 may set the same sensor element drive temperature as the sensor element drive temperature set up to that point in time. The temperature setting unit 112 notifies the heater control unit 113 of the thus set sensor element drive temperature.

The heater control unit 113 controls the operation of the heater 70 based on the sensor element drive temperature that the heater control unit 113 has been notified of by the temperature setting unit 112. For example, the heater control unit 113 controls the heater voltage to be applied to the heater power source 77 such that the value of heater resistance (the resistance of the heater element 72) is a value corresponding to the sensor element drive temperature that the heater control unit 113 has been notified of by the temperature setting unit 112. The heater control unit 113 controls the input power (power supply) from the heater power source 77 to the heater 70, and the heater 70 heats the gas sensor element 100 such that the temperature of the gas sensor element 100 is the sensor element drive temperature set by the temperature setting unit 112.

In this configuration, if it is determined that the NOₓ concentration in the measurement target gas is lower than the reference concentration, the sensor element drive temperature that the gas sensor element 100 is to reach as a result of being heated by the heater 70 is set to be lower than the sensor element drive temperature set if it is determined that the NOₓ concentration is higher than the reference concentration. In other words, the sensor element drive temperature is lowered if the NOₓ concentration is lower than the reference concentration, and is raised if the NOₓ concentration is higher than the reference concentration.

Therefore, when the NOₓ concentration is low, the change in the offset value can be suppressed by lowering the sensor element drive temperature. When the NOₓ concentration is high, the NOₓ decomposition reaction in the measurement target gas can be prevented from being suppressed by raising the sensor element drive temperature. Further, when the NOₓ concentration is high, it is also possible to suppress the change in output in the case of using the gas sensor S for a long period of time by raising the sensor element drive temperature. Accordingly, the gas sensor S can realize highly accurate concentration measurement in both environments where the NOₓ concentration in the measurement target gas is high and where the concentration is low.

Slope of Cell Resistance of Measurement Pump Cell with Respect to Input Power

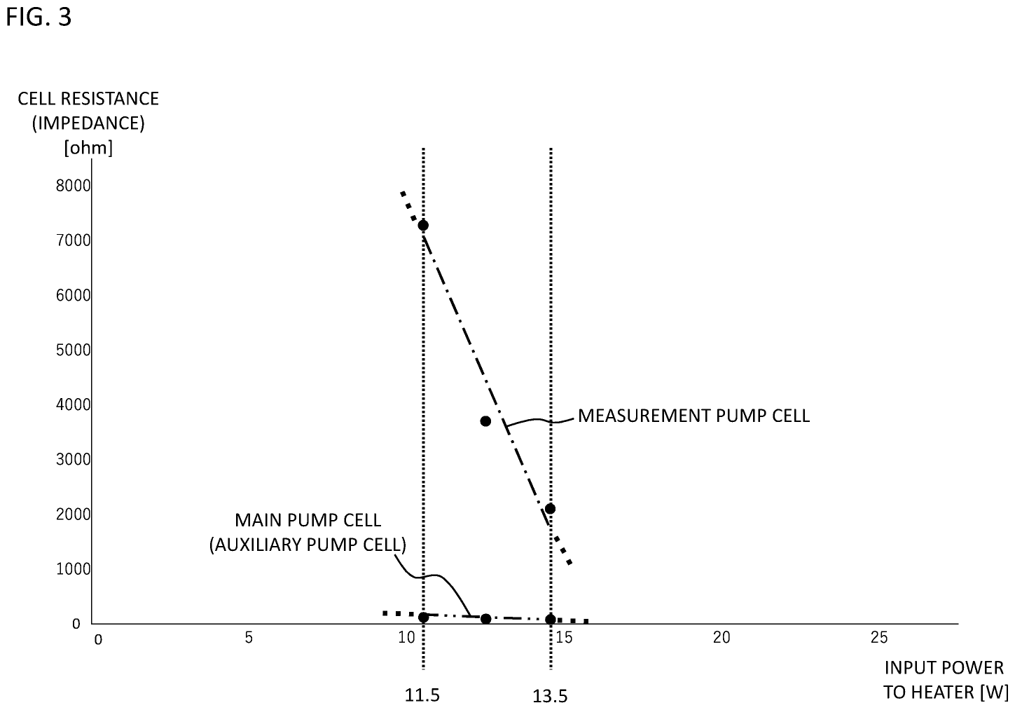

FIG. 3 shows an example regarding the conception of the slope of the cell resistance of the measurement pump cell 41 with respect to input power (power supply) to the heater 70 in the gas sensor S. In the gas sensor S, the slope of the cell resistance (impedance) [ohm] of the measurement pump cell 41 with respect to the input power [W] to the heater 70 is about 2600 [ohm/W] with the input power to the heater 70 in the range from 11.5 to 13.5. That is, the impedance between the measurement electrode 44 and the outer pump electrode 23 with respect to the input power to the heater 70 is about 2600 [ohm/W] with the input power in the range from 11.5 to 13.5. Note that, for example, the cell resistance can be obtained by measuring an I-V curve in the atmosphere and calculating the slope from the current value when the voltage is swept at 5 mV/s between 0 and 50 mV. The slope of the cell resistance with respect to the input power to the heater 70 refers to, for example, the slope of the cell resistance with respect to the input power to the heater 70 in the atmosphere.

Here, the inventor confirmed through experiments that it is desirable that the slope of the cell resistance of the measurement pump cell 41 with respect to the input power to the heater 70 is 200 [ohm/W] or more and 5000 [ohm/W] or less.

That is, if the value of the cell resistance of the measurement pump cell 41 significantly changes in response to the change in the input power to the heater 70, the (change in) input power required to control the value of the cell resistance of the measurement pump cell 41 can be reduced. That is, the value of the cell resistance of the measurement pump cell 41 can be controlled with a small amount of (change in) input power to the heater 70 by increasing the slope of the cell resistance of the measurement pump cell 41 with respect to the input power to the heater 70. Further, the inventor confirmed through experiments that it is desirable to set the slope of the cell resistance of the measurement pump cell 41 with respect to the input power to the heater 70 to 200 [ohm/W] or more in the atmosphere, for example. The value of the cell resistance of the measurement pump cell 41 can be controlled with a small amount of input power by setting the slope of the cell resistance of the measurement pump cell 41 with respect to the input power to the heater 70 to 200 [ohm/W] or more.

The inventor also confirmed from the experiment results and various other perspectives that it is desirable to set the slope of the cell resistance of the measurement pump cell 41 with respect to the input power to the heater 70 to 5000 [ohm/W] or less in the atmosphere, for example. The value of the cell resistance of the measurement pump cell 41 can be controlled with a small amount of input power by setting the slope of the cell resistance of the measurement pump cell 41 with respect to the input power to the heater 70 to 5000 [ohm/W] or less.

Here, in the gas sensor S, the slope of the cell resistance of the measurement pump cell 41 with respect to the input power to the heater 70 is about 2600 [ohm/W] with the input power to the heater 70 in the range from 11.5 to 13.5, as mentioned above. Therefore, the gas sensor S can control the value of the cell resistance of the measurement pump cell 41 while reducing the input power to the heater 70.

Note that the value of the cell resistance of the measurement pump cell 41 may alternatively be measured using the following impedance detection circuit, for example. That is, the value of the cell resistance of the measurement pump cell 41 may be measured using an impedance detection circuit that is inserted and connected between the measurement electrode 44 of the measurement pump cell 41 and the outer pump electrode 23 and detects the impedance between the measurement electrode 44 and the outer pump electrode 23. This impedance detection circuit may include an alternating-current generation circuit that supplies an alternating current between the measurement electrode 44 and the outer pump electrode 23, and a signal detection circuit that detects a voltage signal at a level corresponding to the impedance generated therebetween due to the alternating current supplied therebetween. This signal detection circuit can be constituted by a filter circuit (e.g., a low-pass filter, a band pass filter, etc.) that converts an alternating-current signal generated between the measurement electrode 44 and the outer pump electrode 23 to a voltage signal at a level corresponding to the impedance between the measurement electrode 44 and the outer pump electrode 23.

Relationship Between Slope of Cell Resistance of Measurement Pump Cell and Slope of Cell Resistance of Adjustment Pump Cell in Terms of which is Larger/Smaller

In the gas sensor S, the slope of cell resistance (impedance) [ohm] of the main pump cell 21 with respect to the input power [W] to the heater 70 is about 11 [ohm/W] with the input power to the heater 70 in the range from 11.5 to 13.5.

In the gas sensor S, the slope of the cell resistance of the measurement pump cell 41 with respect to the input power to the heater 70 is about 2600 [ohm/W] with the input power to the heater 70 in the range from 11.5 to 13.5, as mentioned above. Accordingly, in the gas sensor S, the slope of the cell resistance of the measurement pump cell 41 with respect to the input power to the heater 70 is larger than the slope of the cell resistance of the main pump cell 21 with respect to the input power to the heater 70. In other words, in the gas sensor S, the slope of the cell resistance of the measurement pump cell 41 with respect to the input power to the heater 70 is large compared to the slope of the cell resistance of the main pump cell 21 with respect to the input power to the heater 70. Also, the slope of the cell resistance of the main pump cell 21 with respect to the input power to the heater 70 is small compared to the slope of the cell resistance of the measurement pump cell 41 with respect to the input power to the heater 70.

Adopting this configuration enables the gas sensor S to control the value of the cell resistance of the measurement pump cell 41 with a small amount of input power, and does not make the temperature of the main pump cell 21 unnecessarily high or low.

As mentioned above, the value of the cell resistance of the measurement pump cell 41 can be controlled with a small amount of (change in) input power to the heater 70 by increasing the slope of the cell resistance of the measurement pump cell 41 with respect to the input power to the heater 70.

In addition, reducing the slope of the cell resistance of the main pump cell 21 with respect to the input power to the heater 70 facilitates favorable control of the temperature of the main pump cell 21, thus enabling control of the NOₓ decomposition reaction in the measurement target gas.

That is, if the temperature of the main pump cell 21 is unnecessarily high, reaction between NOₓ in the measurement target gas and the inner pump electrode 22 increases in the main pump cell 21, resulting in excessively promoting the NOₓ decomposition reaction in the measurement target gas. Meanwhile, if the temperature of the main pump cell 21 is unnecessarily low, the pump voltage Vp0 at the main pump cell 21 increases, resulting in excessively promoting the NOₓ decomposition reaction in the measurement target gas.

In contrast, the gas sensor S can favorably control the temperature of the main pump cell 21 since the slope of the cell resistance of the main pump cell 21 with respect to the input power to the heater 70 is small. In the gas sensor S, which favorably controls the temperature of the main pump cell 21, the temperature of the main pump cell 21 does not become unnecessarily high or low, and the NOₓ decomposition reaction in the measurement target gas can be prevented from being excessively promoted.

Accordingly, the gas sensor S can control the value of the cell resistance of the measurement pump cell 41 with a small amount of input power to the heater 70, and can also favorably control the temperature of the main pump cell 21 to prevent the NOₓ decomposition reaction from being excessively promoted.

### Ratio Between Slope of Cell Resistance of Measurement Pump Cell and Slope of Cell Resistance of Adjustment Pump Cell

In the gas sensor S, the slope of the cell resistance of the measurement pump cell 41 with respect to the input power to the heater 70 is about 2600 [ohm/W], and the slope of the cell resistance of the main pump cell 21 with respect to the input power to the heater 70 is about 11 [ohm/W], as illustrated in FIG. 3. Accordingly, in the gas sensor S, the slope of the cell resistance of the measurement pump cell 41 with respect to the input power to the heater 70 is about 236 times the slope of the cell resistance of the main pump cell 21 with respect to the input power to the heater 70.

Here, the inventor confirmed through experiments that it is desirable to make the slope of the cell resistance of the measurement pump cell 41 with respect to the input power to the heater 70 10 to 1000 times the slope of the cell resistance of the main pump cell 21 with respect to the input power to the heater 70.

As mentioned above, it is desirable that the slope of the cell resistance of the measurement pump cell 41 with respect to the input power to the heater 70 is larger than the slope of the cell resistance of the main pump cell 21 with respect to the input power to the heater 70. The inventor confirmed through experiments that it is desirable to make the slope of the cell resistance of the measurement pump cell 41 with respect to the input power to the heater 70 10 to 1000 times the slope of the cell resistance of the main pump cell 21 with respect to the input power to the heater 70. Accordingly, the gas sensor S can control the value of the cell resistance of the measurement pump cell 41 with a small amount of input power to the heater 70 and can also favorably control the temperature of the main pump cell 21 to prevent the NOₓ decomposition reaction from being excessively promoted.

Note that, as with the value of the cell resistance of the measurement pump cell 41, the value of the cell resistance of the main pump cell 21 may be measured using, for example, an impedance detection circuit that is inserted and connected between the inner pump electrode 22 and the outer pump electrode 23 of the main pump cell 21 and detects the impedance therebetween.

The above description is of an example where the main pump cell 21 is the adjustment pump cell whose slope of cell resistance with respect to the input power to the heater 70 is smaller than the slope of the cell resistance of the measurement pump cell 41 with respect to the input power to the heater 70. However, the auxiliary pump cell 50 may be the adjustment pump cell whose slope of cell resistance with respect to the input power to the heater 70 is smaller than the slope of the cell resistance of the measurement pump cell 41 with respect to the input power to the heater 70. For example, the slope of the cell resistance of the measurement pump cell 41 with respect to the input power to the heater 70 may be made larger than the slope of the cell resistance of the auxiliary pump cell 50 with respect to the input power to the heater 70. Also, the slope of the cell resistance of the measurement pump cell 41 with respect to the input power to the heater 70 may be made larger than the slopes of the cell resistance of both the main pump cell 21 and the auxiliary pump cell 50 with respect to the input power to the heater 70. That is, the slope of the cell resistance of the measurement pump cell 41 with respect to the input power to the heater 70 need only be larger than the slope of the cell resistance of at least either the main pump cell 21 or the measurement pump cell 41 with respect to this input power.

Similarly, the auxiliary pump cell 50 may be the adjustment pump cell whose slope of cell resistance with respect to the input power to the heater 70 is one-thousandth to one-tenth of the slope of the cell resistance of the measurement pump cell 41 with respect to the input power to the heater 70. For example, the slope of the cell resistance of the measurement pump cell 41 with respect to the input power to the heater 70 may be 10 to 1000 times the slope of the cell resistance of the auxiliary pump cell 50 with respect to the input power to the heater 70. Also, the slope of the cell resistance of the measurement pump cell 41 with respect to the input power to the heater 70 may be 10 to 1000 times the slope of the cell resistance of the main pump cell 21 with respect to the input power to the heater 70 and 10 to 1000 times the slope of the cell resistance of the auxiliary pump cell 50 with respect to the input power to the heater 70. That is, the slope of the cell resistance of the measurement pump cell 41 with respect to the input power to the heater 70 need only be 10 to 1000 times the slope of the cell resistance of at least either the main pump cell 21 or the measurement pump cell 41 with respect to this input power.

As with the value of the cell resistance of the measurement pump cell 41, the value of the cell resistance of the auxiliary pump cell 50 may also be measured using, for example, an impedance detection circuit that is inserted and connected between the auxiliary pump electrode 51 and the outer pump electrode 23 (or an appropriate electrode on the outer side of the gas sensor element 100) of the auxiliary pump cell 50 and detects the impedance therebetween.

### Features

As described above, the gas sensor S according to this embodiment includes the gas sensor element 100, the determination unit 111, and the temperature setting unit 112. The gas sensor element 100 is a sensor element constituted by six oxygen ion-conductive solid electrolyte layers. The gas sensor element 100 includes the internal space (i.e., the measurement target gas flow section 7) into which a measurement target gas is introduced, the measurement pump cell 41, and the heater 70 (heater unit). The measurement pump cell 41 is an electrochemical pump cell constituted by the measurement electrode 44 provided in the measurement target gas flow section 7, the outer pump electrode 23 provided in a region different from the measurement target gas flow section 7, and solid electrolyte layers (the second solid electrolyte layer 6, the spacer layer 5, and the first solid electrolyte layer 4) that are present between the measurement electrode 44 and the outer pump electrode 23. The heater 70 is embedded in the gas sensor element 100 and heats the gas sensor element 100 to a specific temperature (sensor element drive temperature). The determination unit 111 determines whether the NOₓ concentration in the measurement target gas is higher or lower than a predetermined concentration (reference concentration), based on the output of the measurement pump cell 41 (e.g., the pump current Ip2 in the measurement pump cell 41). The temperature setting unit 112 sets the specific temperature (sensor element drive temperature) that the gas sensor element 100 is to reach as a result of being heated by the heater 70, based on the result (determination result) of the determination by the determination unit 111. Specifically, if the determination unit 111 determines that the NOₓ concentration is lower than the reference concentration, the temperature setting unit 112 sets the specific temperature to be lower than the specific temperature set if the determination unit 111 determines that the NOₓ concentration is higher than the reference concentration.

A gas sensor control method according to this embodiment is a method for controlling a gas sensor that includes the gas sensor element 100, and is an information processing method for executing a determination step and a temperature setting step. In the determination step, it is determined whether the NOₓ concentration in the measurement target gas is higher or lower than a predetermined concentration (reference concentration), based on the output (e.g., the pump current Ip2) of the measurement pump cell 41. In the temperature setting step, if it is determined in the determination step that the NOₓ concentration is lower (than the reference concentration), the specific temperature (sensor element drive temperature) that the gas sensor element 100 is to reach as a result of being heated by the heater 70 is made lower than the specific temperature set if it is determined that the NOₓ concentration is higher.

In this configuration, if it is determined that the NOₓ concentration in the measurement target gas is lower than the reference concentration, the sensor element drive temperature that the gas sensor element 100 is to reach as a result of being heated by the heater 70 is set to be lower than the sensor element drive temperature set if it is determined that the NOₓ concentration is higher than the reference concentration. In other words, the sensor element drive temperature is lowered if the NOₓ concentration is lower than the reference concentration, and is raised if the NOₓ concentration is higher than the reference concentration.

Therefore, the gas sensor S can lower the sensor element drive temperature to suppress the change in the offset value when the NOₓ concentration is low, and can raise the sensor element drive temperature to prevent the NOₓ decomposition reaction in the measurement target gas from being suppressed when the NOₓ concentration is high. Also, in the gas sensor S, the change in the output in the case of using the gas sensor S for a long period of time can also be suppressed by raising the sensor element drive temperature when the NOₓ concentration is high.

Accordingly, the gas sensor S can realize highly accurate concentration measurement in both environments where the NOₓ concentration in the measurement target gas is high and where the NOₓ concentration is low.

In the gas sensor according to this embodiment, the gas sensor element 100 may include the adjustment pump cell, i.e., at least either the main pump cell 21 or the auxiliary pump cell 50. The adjustment pump cell included in the gas sensor element 100 is an electrochemical pump cell constituted by the inner pump electrode (the inner pump electrode 22 or the auxiliary pump electrode 51) facing the internal space (i.e., the measurement target gas flow section 7) in the gas sensor element 100, the outer pump electrode 23 or the third electrode in contact with at least one of the solid electrolyte layers 1 to 6 and exposed to the external space, and a solid electrolyte layer that is present between the inner pump electrode and the outer pump electrode 23 or the third electrode.

Specifically, the main pump cell 21 is an electrochemical pump cell constituted by the inner pump electrode 22 facing the measurement target gas flow section 7, the outer pump electrode 23, and the second solid electrolyte layer 6 held between the inner pump electrode 22 and the outer pump electrode 23. The auxiliary pump cell 50 is an electrochemical pump cell constituted by the auxiliary pump electrode 51, the outer pump electrode 23 (or an appropriate electrode on the outer side of the gas sensor element 100 that is in contact with at least one of the solid electrolyte layers 1 to 6), and a solid electrolyte layer (e.g., the second solid electrolyte layer 6) that is held therebetween.

The measurement target gas out of which oxygen has been pumped in the adjustment pump cell (i.e., at least either the main pump cell 21 or the auxiliary pump cell 50) is introduced into the measurement pump cell 41.

Here, the slope of the cell resistance of the measurement pump cell 41 with respect to the input power to the heater 70 may be larger than the slope of the cell resistance of the adjustment pump cell (i.e., at least either the main pump cell 21 or the auxiliary pump cell 50) with respect to the input power to the heater 70.

Adopting this configuration enables the gas sensor according to one aspect of the present invention to control the value of the cell resistance of the measurement pump cell 41 with a small amount of input power to the heater 70. Further, since the gas sensor according to one aspect of the present invention adopts this configuration, the temperature of the adjustment pump cell (i.e., at least either the main pump cell 21 or the auxiliary pump cell 50) does not become unnecessarily high or low.

That is, the value of the cell resistance of the measurement pump cell 41 can be controlled with a small amount of (change in) input power to the heater 70 by increasing the slope of the cell resistance of the measurement pump cell 41 with respect to the input power to the heater 70.

Also, reducing the slope of the cell resistance of the adjustment pump cell (i.e., at least either the main pump cell 21 or the auxiliary pump cell 50) with respect to the input power to the heater 70 facilitates favorable control of the temperature of the adjustment pump cell, and enables control of the NOₓ decomposition reaction in the measurement target gas.

That is, if the temperature of the adjustment pump cell (i.e., at least either the main pump cell 21 or the auxiliary pump cell 50) is unnecessarily high, reaction between NOₓ in the measurement target gas and the inner pump electrode (at least either the inner pump electrode 22 or the auxiliary pump electrode 51) increases in the adjustment pump cell. Consequently, the NOₓ decomposition reaction in the measurement target gas is excessively promoted. Meanwhile, if the temperature of the adjustment pump cell (i.e., at least either the main pump cell 21 or the auxiliary pump cell 50) is unnecessarily low, the pump voltage (at least either the pump voltage Vp0 or the voltage Vp1) at the adjustment pump cell increases, thus excessively promoting the NOₓ decomposition reaction in the measurement target gas.

In contrast, in the gas sensor according to one aspect of the present invention, the slope of the cell resistance of the adjustment pump cell (i.e., at least either the main pump cell 21 or the auxiliary pump cell 50) with respect to the input power to the heater 70 is small. This enables favorable control of the temperature of the adjustment pump cell. The gas sensor according to one aspect of the present invention favorably controls the temperature of the adjustment pump cell (i.e., at least either the main pump cell 21 or the auxiliary pump cell 50). Therefore, the temperature of the adjustment pump cell does not become unnecessarily high or low, and the NOₓ decomposition reaction in the measurement target gas can be prevented from being excessively promoted.

Accordingly, the gas sensor according to one aspect of the present invention can control the value of the cell resistance of the measurement pump cell 41 with a small amount of input power to the heater 70, and can also favorably control the temperature of the adjustment pump cell (i.e., at least either the main pump cell 21 or the auxiliary pump cell 50) to prevent the NOₓ decomposition reaction from being excessively promoted.

In the gas sensor according to the above aspect, the slope of the cell resistance of the measurement pump cell 41 with respect to the input power to the heater 70 may be 10 to 1000 times the slope of the cell resistance of the adjustment pump cell (i.e., at least either the main pump cell 21 or the auxiliary pump cell 50) with respect to the input power to the heater 70.

As mentioned above, it is desirable that the slope of the cell resistance of the measurement pump cell 41 with respect to the input power to the heater 70 is larger than the slope of the cell resistance of the adjustment pump cell (i.e., at least either the main pump cell 21 or the auxiliary pump cell 50) with respect to the input power to the heater 70. The inventor confirmed through experiments that it is desirable to make the slope of the cell resistance of the measurement pump cell 41 with respect to the input power to the heater 70 10 to 1000 times the slope of the cell resistance of the adjustment pump cell (i.e., at least either the main pump cell 21 or the auxiliary pump cell 50) with respect to the input power to the heater 70.

### Variations

Although an embodiment of the present invention has been described above, the description of the above embodiment is merely an illustration of the invention in all respects. Various improvements and variations may be made to the above embodiment. The constituent elements of the above embodiment may be omitted, replaced, and added as appropriate. The shape and dimensions of each constituent element of the above embodiment may be changed as appropriate, as per the mode of implementation. For example, the following changes are possible. Note that, in the following, the same constituent elements as those of the above embodiment are assigned the same reference numerals, and the description of the same features as the above embodiment is omitted as appropriate. The following variations can be combined as appropriate.

### (I) Slope of Cell Resistance of Measurement Pump Cell

The above description is of the gas sensor S in which the slope of the cell resistance of the measurement pump cell 41 (the impedance between the measurement electrode 44 and the outer pump electrode 23) with respect to the input power to the heater 70 is about 2600 [ohm/W]. However, it is not essential that the slope of the cell resistance of the measurement pump cell 41 with respect to the input power to the heater 70 is about 2600 [ohm/W]. As mentioned above, it is desirable that the slope of the cell resistance of the measurement pump cell 41 with respect to the input power to the heater 70 is 200 [ohm/W] or more and 5000 [ohm/W] or less.

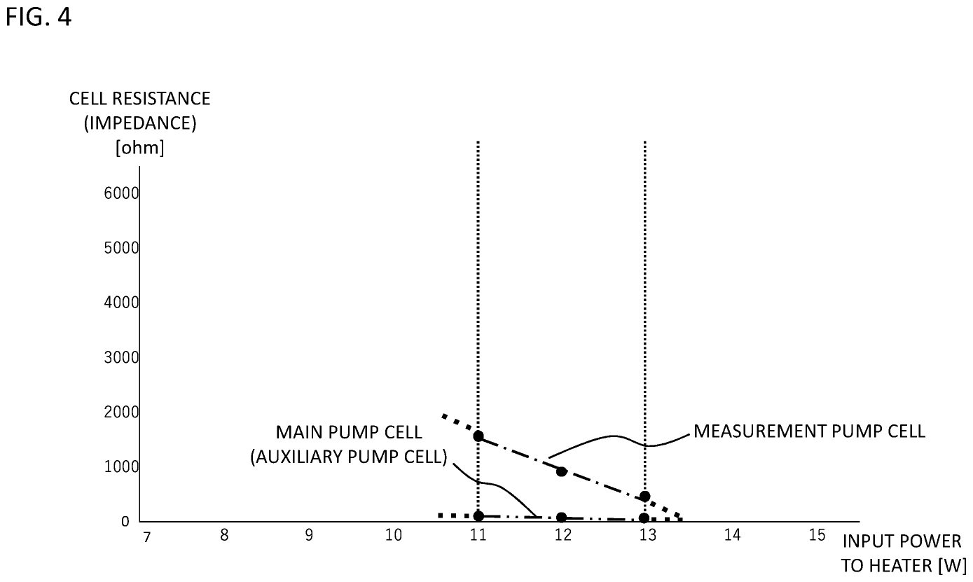

FIG. 4 shows an example regarding the conception of the slope of the cell resistance of the measurement pump cell 41 with respect to the input power (power supply) to the heater 70 in a gas sensor S1 according to a variation. In the gas sensor S1, the slope of the cell resistance (impedance) of the measurement pump cell 41 with respect to the input power to the heater 70 is about 600 [ohm/W] with the input power to the heater 70 in the range from 11.5 to 13.5. That is, in the gas sensor S1, the impedance between the measurement electrode 44 and the outer pump electrode 23 with respect to the input power to the heater 70 takes a value that is 200 [ohm/W] or more and 5000 [ohm/W] or less; specifically, about 600 [ohm/W]. Therefore, the gas sensor S1 can control the value of the cell resistance of the measurement pump cell 41 while reducing the input power to the heater 70.

(II) Relationship Between Slopes of Cell Resistance in Terms of which is Larger/Smaller

The above description is of the gas sensor S in which the slope of the cell resistance (impedance) of the main pump cell 21 with respect to the input power to the heater 70 is about 11 [ohm/W] with the input power to the heater 70 in the range from 11.5 to 13.5. However, it is not essential that the slope of the cell resistance of the main pump cell 21 with respect to the input power to the heater 70 is about 11 [ohm/W]. As mentioned above, it is desirable that the slope of the cell resistance of the measurement pump cell 41 with respect to the input power to the heater 70 is made larger than the slope of the cell resistance of the main pump cell 21 with respect to the input power to the heater 70.

In the gas sensor S1, the slope of the cell resistance of the main pump cell 21 with respect to the input power [W] to the heater 70 is about 10 [ohm/W] with the input power to the heater 70 in the range from 11.5 to 13.5, as illustrated in FIG. 4. Further, in the gas sensor S1, the slope of the cell resistance of the measurement pump cell 41 with respect to the input power to the heater 70 is about 600 [ohm/W] with the input power to the heater 70 in the range from 11.5 to 13.5. Therefore, in the gas sensor S1, the slope of the cell resistance of the measurement pump cell 41 with respect to the input power to the heater 70 is larger than the slope of the cell resistance of the main pump cell 21 with respect to the input power to the heater 70.

Accordingly, the gas sensor S1 can control the value of the cell resistance of the measurement pump cell 41 with a small amount of input power to the heater 70, and can also favorably control the temperature of the main pump cell 21 to prevent the NOₓ decomposition reaction from being excessively promoted.

Note that, in the gas sensor S1, the auxiliary pump cell 50 may be the adjustment pump cell whose slope of cell resistance with respect to the input power to the heater 70 is smaller than the slope of the cell resistance of the measurement pump cell 41 with respect to the input power to the heater 70. In other words, in the gas sensor S1, the slope of the cell resistance of the measurement pump cell 41 with respect to the input power to the heater 70 may be made larger than the slope of the cell resistance of the auxiliary pump cell 50 with respect to the input power to the heater 70. Also, the slope of the cell resistance of the measurement pump cell 41 with respect to the input power to the heater 70 may be made larger than the slopes of the cell resistance of both the main pump cell 21 and the auxiliary pump cell 50 with respect to the input power to the heater 70. In the gas sensor S1, the slope of the cell resistance of the measurement pump cell 41 with respect to the input power to the heater 70 need only be larger than the slope of the cell resistance of at least either the main pump cell 21 or the auxiliary pump cell 50 with respect to this input power.

### (III) Ratio Between Slopes of Cell Resistance

The above description is of the gas sensor S in which the slope of the cell resistance of the measurement pump cell 41 with respect to the input power to the heater 70 is about 236 times the slope of the cell resistance of the main pump cell 21 with respect to the input power to the heater 70. However, it is not essential that the slope of the cell resistance of the measurement pump cell 41 with respect to the input power to the heater 70 is about 236 times the slope of the cell resistance of the main pump cell 21 with respect to the input power to the heater 70. As mentioned above, it is desirable that the slope of the cell resistance of the measurement pump cell 41 with respect to the input power to the heater 70 is 10 to 1000 times the slope of the cell resistance of the main pump cell 21 with respect to the input power to the heater 70.

In the gas sensor S1, the slope of the cell resistance of the measurement pump cell 41 with respect to the input power to the heater 70 is about 600 [ohm/W], and the slope of the cell resistance of the main pump cell 21 with respect to the input power to the heater 70 is about 10 [ohm/W], as illustrated in FIG. 4. Accordingly, in the gas sensor S1, the slope of the cell resistance of the measurement pump cell 41 with respect to the input power to the heater 70 is about 60 times the slope of the cell resistance of the main pump cell 21 with respect to the input power to the heater 70. In other words, in the gas sensor S1, the slope of the cell resistance of the measurement pump cell 41 with respect to the input power to the heater 70 takes a value that is 10 to 1000 times the slope of the cell resistance of the main pump cell 21 with respect to the input power to the heater 70. Accordingly, the gas sensor S1 can control the value of the cell resistance of the measurement pump cell 41 with a small amount of input power to the heater 70 and can also favorably control the temperature of the main pump cell 21 to prevent the NOₓ decomposition reaction from being excessively promoted.

Note that, in the gas sensor S1, the auxiliary pump cell 50 may be the adjustment pump cell whose slope of cell resistance with respect to the input power to the heater 70 is one-thousandth to one-tenth of the slope of the cell resistance of the measurement pump cell 41 with respect to the input power to the heater 70. For example, the slope of the cell resistance of the measurement pump cell 41 with respect to the input power to the heater 70 may be 10 to 1000 times the slope of the cell resistance of the auxiliary pump cell 50 with respect to the input power to the heater 70. Also, the slope of the cell resistance of the measurement pump cell 41 with respect to the input power to the heater 70 may be 10 to 1000 times the slope of the cell resistance of the main pump cell 21 with respect to the input power to the heater 70 and 10 to 1000 times the slope of the cell resistance of the auxiliary pump cell 50 with respect to the input power to the heater 70. In the gas sensor S1, the slope of the cell resistance of the measurement pump cell 41 with respect to the input power to the heater 70 need only be 10 to 1000 times the slope of the cell resistance of at least either the main pump cell 21 or the auxiliary pump cell 50 with respect to this input power.

### (IV) Control of Cell Resistance of Measurement Pump Cell 41

The above description is of the gas sensor S that includes the determination unit 111 and the temperature setting unit 112, i.e., the gas sensor S that executes sensor element drive temperature setting processing. However, the gas sensor according to this embodiment may also execute constant-impedance control in addition to the sensor element drive temperature setting processing.

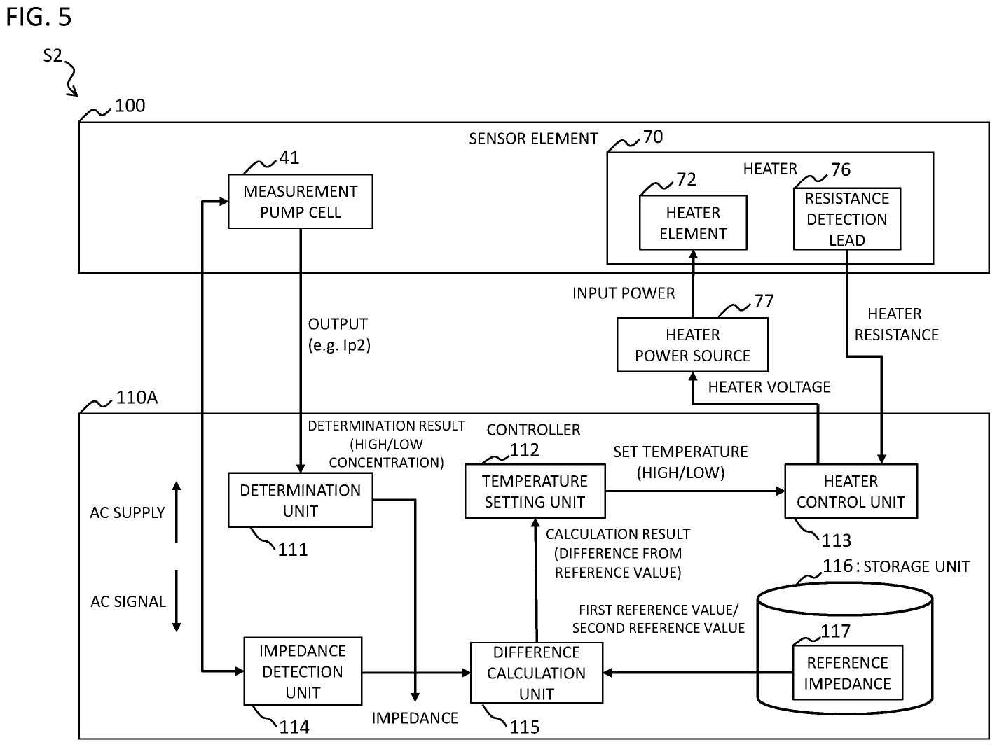

FIG. 5 shows an overview of the constant-impedance control in a gas sensor S2 according to a variation. As illustrated in FIG. 5, the gas sensor S2 includes the gas sensor element 100 and a controller 110A. Note that the gas sensor S2 has constituent elements with common operations and functions to the constituent elements of the gas sensor S that has been described with reference to FIG. 1 and other figures. Those constituent elements are assigned the same reference numerals as those of the corresponding constituent elements shown in FIG. 1 and other figures, and a detailed description thereof is omitted unless necessary.

The controller 110A controls operation of each part of the gas sensor S2, identifies the NOₓ concentration based on the pump current Ip2 flowing through the gas sensor element 100, and heats the gas sensor element 100 to a specific temperature (sensor element drive temperature) using the heater 70. The controller 110A is realized by a general-purpose or dedicated computer, and includes, as functional constituent elements realized by its CPU, memory, or the like, the detection unit 111, the temperature setting unit 112, the heater control unit 113, an impedance detection unit 114, a difference calculation unit 115, and a storage unit 116, as illustrated in FIG. 5. That is, the controller 110A includes, as functional components, the determination unit 111, the temperature setting unit 112, and the heater control unit 113, similarly to the above-described controller 110. The controller 110A also includes, as functional components, the impedance detection unit 114, the difference calculation unit 115, and the storage unit 116. Note that, if the gas sensor S2 is for detecting and measuring NOₓ contained in exhaust gas from an automobile engine, and the gas sensor element 100 is attached to an exhaust path, some or all of the functions of the controller 110A may be realized by ECUs (Electronic Control Units) installed in the automobile.

The functional blocks in the controller 110A in FIG. 5 include the determination unit 111, the temperature setting unit 112, the heater control unit 113, the impedance detection unit 114, the difference calculation unit 115, and the storage unit 116. However, the controller 110A may also include any functional blocks other than these functional blocks. For example, the controller 110A may include functional blocks for detecting NOₓ and calculating the concentration thereof, or for other purposes. Specifically, the controller 110A may also include a functional block for controlling operation of each pump cell, a functional block for calculating the NOₓ concentration, a functional block for comprehensively controlling operation of each part of the controller 110A, or the like.

Similarly to the determination unit 111 included in the controller 110, the determination unit 111 included in the controller 110A determines whether the NOₓ concentration in the measurement target gas is higher or lower than a predetermined concentration (reference concentration), based on the output of the measurement pump cell 41 (determination step). The determination unit 111 included in the controller 110A notifies the difference calculation unit 115 of the result (determination result) of the determination performed using the output of the measurement pump cell 41, i.e., whether the NOₓ concentration in the measurement target gas is higher or lower than the reference concentration.

The impedance detection unit 114 measures (detects) the value of the cell resistance (impedance) of the measurement pump cell 41, and notifies the difference calculation unit 115 of the measured value of the cell resistance of the measurement pump cell 41. The impedance detection unit 114 may, for example, supply an alternating current between the measurement electrode 44 and the outer pump electrode 23 of the measurement pump cell 41, and convert an alternating-current signal generated therebetween to a voltage signal at a level corresponding to the impedance therebetween.

Specifically, the impedance detection unit 114 may be an impedance detection circuit that is inserted and connected between the measurement electrode 44 and the outer pump electrode 23 of the measurement pump cell 41 and detects the impedance between the measurement electrode 44 and the outer pump electrode 23. The impedance detection unit 114 may include an alternating-current generation circuit for supplying an alternating current between the measurement electrode 44 and the outer pump electrode 23, and a signal detection circuit for detecting a voltage signal at a level corresponding to the impedance generated therebetween due to the supply of the alternating current therebetween. The signal detection circuit included in the impedance detection unit 114 may be constituted by a filter circuit (e.g., a low-pass filter, a band pass filter etc.) that converts an alternating-current signal generated between the measurement electrode 44 and the outer pump electrode 23 to a voltage signal at a level corresponding to the impedance between the measurement electrode 44 and the outer pump electrode 23.

The difference calculation unit 115 calculates a difference between the value of the cell resistance of the measurement pump cell 41 and a first reference value or a second reference value using the determination result that the difference calculation unit 115 has been notified of by the determination unit 111 and the value of the cell resistance of the measurement pump cell 41 that the difference calculation unit 115 has been notified of by the impedance detection unit 114 (difference calculation step). The difference calculation unit 115 notifies the temperature setting unit 112 of the calculated difference between the value of the cell resistance of the measurement pump cell 41 and the reference value (the first reference value or the second reference value).

Specifically, if the determination unit 111 notifies the difference calculation unit 115 of the determination result that the NOₓ concentration is lower than the reference concentration, the difference calculation unit 115 references the reference impedance 117 in the storage unit 116 and obtains the first reference value. The difference calculation unit 115 then calculates a difference between the obtained first reference value and the value of the cell resistance of the measurement pump cell 41 that the difference calculation unit 115 has been notified of by the impedance detection unit 114, and notifies the temperature setting unit 112 of the calculated difference. The first reference value is a value that is preset as a value of the cell resistance that the measurement pump cell 41 is to exhibit when the NOₓ concentration is lower than the reference concentration.

Also, if the determination unit 111 notifies the difference calculation unit 115 of the determination result that the NOₓ concentration is higher than the reference concentration, the difference calculation unit 115 references the reference impedance 117 in the storage unit 116 and obtains the second reference value. The difference calculation unit 115 then calculates a difference between the obtained second reference value and the value of the cell resistance of the measurement pump cell 41 that the difference calculation unit 115 has been notified of by the impedance detection unit 114, and notifies the temperature setting unit 112 of the calculated difference. The second reference value is a value that is preset as a value of the cell resistance that the measurement pump cell 41 is to exhibit when the NOₓ concentration is higher than the reference concentration.

If the determination unit 111 notifies the difference calculation unit 115 of the determination result that the NOₓ concentration is equal to the reference concentration, the difference calculation unit 115 may give the temperature setting unit 112 an instruction to maintain the sensor element drive temperature set up to that point in time by the temperature setting unit 112.

The temperature setting unit 112 sets the sensor element drive temperature (temperature setting step, constant-impedance control step) based on the difference between the value of the cell resistance of the measurement pump cell 41 and the reference value (the first reference value or the second reference value) that the temperature setting unit 112 has been notified of by the difference calculation unit 115. Specifically, the temperature setting unit 112 sets the sensor element drive temperature so as to reduce the difference between the value of the cell resistance of the measurement pump cell 41 and the reference value (the first reference value or the second reference value). In other words, the temperature setting unit 112 sets the sensor element drive temperature such that the value of the cell resistance of the measurement pump cell 41 is equal to the reference value (the first reference value or the second reference value). The temperature setting unit 112 then notifies the heater control unit 113 of the set sensor element drive temperature.

For example, if the value of the cell resistance of the measurement pump cell 41 is smaller than the reference value (the first reference value or the second reference value), the temperature setting unit 112 lowers the sensor element drive temperature set up to that point in time so as to increase the value of the cell resistance of the measurement pump cell 41 and make this value equal to the reference value. The temperature setting unit 112 notifies the heater control unit 113 of the new sensor element drive temperature that has been lowered such that the value of the cell resistance of the measurement pump cell 41 is equal to the reference value.

For example, if the value of the cell resistance of the measurement pump cell 41 is larger than the reference value (the first reference value or the second reference value), the temperature setting unit 112 raises the sensor element drive temperature set up to that point in time so as to reduce the value of the cell resistance of the measurement pump cell 41 and make this value equal to the reference value. The temperature setting unit 112 notifies the heater control unit 113 of the new sensor element drive temperature that has been raised such that the value of the cell resistance of the measurement pump cell 41 is equal to the reference value.

Similarly to the heater control unit 113 included in the controller 110, the heater control unit 113 included in the controller 110A controls the operation of the heater 70 based on the sensor element drive temperature that the heater control unit 113 has been notified of by the temperature setting unit 112. The heater control unit 113 controls the input power (power supply) from the heater power source 77 to the heater 70, and the heater 70 heats the gas sensor element 100 such that the temperature of the gas sensor element 100 is the sensor element drive temperature that is set by the temperature setting unit 112. The heater control unit 113 may also control a heater voltage applied to the heater power source 77 such that the value of heater resistance (resistance of the heater element 72), which is obtained as a resistance value between the heater resistance detection lead 76 and the heater lead 72a, is a value corresponding to the specific temperature that is set by the temperature setting unit 112.

As described above, if, in the gas sensor S2, it is determined that the NOₓ concentration is lower than the reference concentration, the value of the cell resistance of the measurement pump cell 41 is controlled so as to be the first reference value (first value).

If, for example, the value of the cell resistance of the measurement pump cell 41 is smaller than the first reference value, the controller 110A lowers the sensor element drive temperature and increases the value of the cell resistance of the measurement pump cell 41 so as to make the value of the cell resistance of the measurement pump cell 41 equal to the first reference value. If, for example, the value of the cell resistance of the measurement pump cell 41 is larger than the first reference value, the controller 110A raises the sensor element drive temperature and reduces the value of the cell resistance of the measurement pump cell 41 so as to make the value of the cell resistance of the measurement pump cell 41 equal to the first reference value. If, for example, the value of the cell resistance of the measurement pump cell 41 is equal to the first reference value, the controller 110A may maintain the sensor element drive temperature set up to that point in time and maintain the state where the value of the cell resistance of the measurement pump cell 41 is equal to the first reference value.

If, in the gas sensor S2, it is determined that the NOₓ concentration is higher than the reference concentration, the value of the cell resistance of the measurement pump cell 41 is controlled so as to be the second reference value (second value).

If, for example, the value of the cell resistance of the measurement pump cell 41 is smaller than the second reference value, the controller 110A lowers the sensor element drive temperature and increases the value of the cell resistance of the measurement pump cell 41 so as to make the value of the cell resistance of the measurement pump cell 41 equal to the second reference value. If, for example, the value of the cell resistance of the measurement pump cell 41 is larger than the second reference value, the controller 110A raises the sensor element drive temperature and reduces the value of the cell resistance of the measurement pump cell 41 so as to make the value of the cell resistance of the measurement pump cell 41 equal to the second reference value. If, for example, the value of the cell resistance of the measurement pump cell 41 is equal to the second reference value, the controller 110A may maintain the sensor element drive temperature set up to that point in time and maintain the state where the value of the cell resistance of the measurement pump cell 41 is equal to the second reference value.

As described above, the gas sensor S2 (specifically, the controller 110A) executes the following constant-impedance control step as a temperature setting step of setting the sensor element drive temperature. That is, the controller 110A (specifically, the temperature setting unit 112) executes the constant-impedance control step of setting the sensor element drive temperature so as to make the value of the cell resistance of the measurement pump cell 41 equal to the reference value (the first reference value or the second reference value). Here, the NOₓ concentration in the measurement target gas determines which of the first reference value and the second reference value is to be adopted as the reference value. If the NOₓ concentration is lower than the reference concentration, the first reference value is adopted as the reference value. If the NOₓ concentration is higher than the reference concentration, the second reference value is adopted as the reference value.

In this configuration, the value of the cell resistance of the measurement pump cell 41 is controlled so as to be the first reference value if it is determined that the NOₓ concentration is low, and the value of the cell resistance of the measurement pump cell 41 is controlled so as to be the second reference value if it is determined that the NOₓ concentration is high. Therefore, the gas sensor S2 can prevent a situation where the measurement results change due to the passage of time (e.g., a change in the value of the cell resistance of the measurement pump cell 41) rather than the NOₓ concentration in the measurement target gas.

### (V) Others

In the above embodiment, the laminate of the gas sensor element 100 is constituted by six solid electrolyte layers. However, the number of solid electrolyte layers to constitute the laminate need not be limited to this example, and may be selected as appropriate, as per the mode of implementation.

In the above embodiment, the internal space (i.e., the measurement target gas flow section 7) into which the measurement target gas is introduced is located at a position demarcated by the first solid electrolyte layer 4, the spacer layer 5, and the second solid electrolyte layer 6. However, the location of the measurement target gas flow section 7 need not be limited to this example, and may be selected as appropriate, as per the mode of implementation. The orientations of a first face, a second face, a first pump electrode, a second pump electrode, a first lead, and a second lead may be selected as appropriate in accordance with the configuration of the laminate and the internal space.

In the above embodiment, the measurement target gas flow section 7 has a three-cavity structure. However, the configuration of the measurement target gas flow section 7 need not be limited to this example, and may be selected as appropriate, as per the mode of implementation. In another example, the fourth diffusion control portion 18 and the third internal cavity 19 may be omitted, and thus, the measurement target gas flow section 7 may have a two-cavity structure. In this case, the measurement electrode 44 may be provided at a position separated from the third diffusion control portion 16 on the upper face of the first solid electrolyte layer 4 adjacent to the second internal cavity 17. That is, the measurement target gas flow section 7 may include two hollow spaces into or from which oxygen is pumped, or may include only one such hollow space. In addition, it is not essential, either, for the gas sensor element 100 to include one or more diffusion control portions.

In FIG. 1, both the inner pump electrode 22 and the outer pump electrode 23 are exposed to a space. However, the manner of adjoining a space need not be limited to this mode, and may alternatively be indirectly adjoined via a coating or the like. As another example, the outer pump electrode 23 may be coated with a protection member or the like.

In the above embodiment, the reference gas inlet space 43 is provided. However, the configuration of the gas sensor element 100 need not be limited to this example. In another example, the first solid electrolyte layer 4 may extend to the rear end of the gas sensor element 100, and the reference gas inlet space 43 may be omitted. In this case, the atmosphere inlet layer 48 may extend to the rear end of the gas sensor element 100.

In the above embodiment, the gas sensor element 100 is configured to measure the concentration of nitrogen oxide (NOₓ). However, the gas sensor element of the present invention need not be limited to such a gas sensor element configured to measure the NOₓ concentration. In another example, the gas sensor element of the present invention may be another gas sensor element, such as a gas sensor element configured to measure the oxygen concentration. For example, a gas sensor element for measuring the oxygen concentration can be configured by omitting the auxiliary pump cell and the measurement pump cell and disposing the reference electrode below the main pump electrode in the gas sensor element 100 according to the above embodiment. In this case, the gas sensor element can measure the oxygen concentration in the measurement target gas by pumping out oxygen using the main pump cell.

### Examples

Gas sensors according to the following examples 1 to 4 were produced in order to examine the effects of the present invention. Note that the present invention is not limited to the gas sensors according to the following examples.

TABLE 1

| | SLOPE A OF ADJUSTMENT PUMP | SLOPE B OF MEASUREMENT PUMP | | | | |
|---|---|---|---|---|---|---|
| | CELL RESISTANCE WITH RESPECT | CELL RESISTANCE WITH RESPECT | | EVALU- | EVALU- | EVALU- |
| | TO INPUT POWER TO HEATER | TO INPUT POWER TO HEATER | | ATION | ATION | ATION |
| LEVEL | [ohm/W] | [ohm/W] | B/A | 1 | 2 | 3 |
| EXAMPLE 1 | 11 | 2600 | 236 | −6% | −7% | +4 ppm |
| EXAMPLE 2 | 10 | 600 | 60 | −8% | −9% | +5 ppm |
| EXAMPLE 3 | 20 | 200 | 10 | −10% | −10% | +7 ppm |
| EXAMPLE 4 | 5 | 5000 | 1000 | −5% | −5% | +3 ppm |

The example 1 is the gas sensor S that has been described with reference to FIGS. 1 to 3. In the gas sensor S, the slope A [ohm/W] of the cell resistance of the adjustment pump cell (e.g., the main pump cell 21) with respect to the input power to the heater 70 is about 11, as mentioned above. In the example 1 (gas sensor S), the slope B [ohm/W] of the cell resistance of the measurement pump cell 41 with respect to the input power to the heater 70 is about 2600. Accordingly, in the example 1, the slope B of the cell resistance of the measurement pump cell 41 with respect to the input power to the heater 70 is about 236 times the slope A of the cell resistance of the adjustment pump cell with respect to the input power to the heater 70. That is, B/A is about 236.

The example 2 is the gas sensor S1 that has been described with reference to FIG. 4. In the gas sensor S1, the slope A [ohm/W] of the cell resistance of the adjustment pump cell (e.g., the main pump cell 21) with respect to the input power to the heater 70 is about 10, as mentioned above. In the example 2 (gas sensor S1), the slope B [ohm/W] of the cell resistance of the measurement pump cell 41 with respect to the input power to the heater 70 is about 600. Accordingly, in the example 2, the slope B of the cell resistance of the measurement pump cell 41 with respect to the input power to the heater 70 is about 60 times the slope A of the cell resistance of the adjustment pump cell with respect to the input power to the heater 70. That is, B/A is about 60.

The example 3 is a gas sensor that has the same configuration as the example 1 (gas sensor S) and the example 2 (gas sensor S1) and in which the adjustment pump cell (e.g., the main pump cell 21) and the measurement pump cell 41 satisfy the following conditions. That is, the gas sensor according to the example 3 includes the gas sensor element 100, the determination unit 111, and the temperature setting unit 112. In the example 3, the slope A [ohm/W] of the cell resistance of the adjustment pump cell (e.g., the main pump cell 21) with respect to the input power to the heater 70 is about 20. Also, in the example 3, the slope B [ohm/W] of the cell resistance of the measurement pump cell 41 with respect to the input power to the heater 70 is about 200. Accordingly, in the example 3, the slope B of the cell resistance of the measurement pump cell 41 with respect to the input power to the heater 70 is about 10 times the slope A of the cell resistance of the adjustment pump cell with respect to the input power to the heater 70 That is, B/A is about 10.

The example 4 is a gas sensor that has the same configuration as the example 1 (gas sensor S) and the example 2 (gas sensor S1) and in which the adjustment pump cell (e.g., the main pump cell 21) and the measurement pump cell 41 satisfy the following conditions. That is, the gas sensor according to the example 4 includes the gas sensor element 100, the determination unit 111, and the temperature setting unit 112. In the example 4, the slope A [ohm/W] of the cell resistance of the adjustment pump cell (e.g., the main pump cell 21) with respect to the input power to the heater 70 is about 5. Also, in the example 4, the slope B [ohm/W] of the cell resistance of the measurement pump cell 41 with respect to the input power to the heater 70 is about 5000. Accordingly, in the example 4, the slope B of the cell resistance of the measurement pump cell 41 with respect to the input power to the heater 70 is about 1000 times the slope of the cell resistance of the adjustment pump cell with respect to the input power to the heater 70. That is, B/A is about 1000.

The inventor implemented the following durability test using a diesel engine on the gas sensors according to the examples 1 to 4 and examined changes in parameters (output values etc.) of each gas sensor before and after the implementation of the durability test. That is, the inventor implemented, as the aforementioned durability test, a test in which each of the gas sensors was attached to an exhaust gas pipe of an automobile, and a 40-minute driving pattern was repeated for 2000 hours in a range where the engine speed was 1500 to 3500 rpm and the load torque was 0 to 350 N·m. Note that, in this durability test, the gas temperature ranged from 200 to 600 degrees Celsius, and the NOₓ concentration ranged from 0 to 1500 ppm.

For each of the gas sensors according to the examples 1 to 4, examination was conducted on changes in the NOₓ output in an environment where the NOₓ concentration was high, the NOₓ output in an environment where the O₂ concentration was high, and the offset value before and after the implementation of the durability test. “EVALUATION 1” in Table 1 shows the percentage changes of the NOₓ output in the environment where the NOₓ concentration was high before and after the implementation of the aforementioned durability test that were confirmed for the examples 1 to 4. “EVALUATION 2” in Table 1 shows the percentage changes of the NOₓ output in the environment where the O₂ concentration was high (i.e., the NOₓ concentration is relatively low) before and after the implementation of the aforementioned durability test that were confirmed for the examples 1 to 4. Further, “EVALUATION 3” in Table 1 shows the offset change amounts of the NOₓ output (the amount of change in the offset value) before and after the implementation of the aforementioned durability test that were confirmed for the examples 1 to 4.

Specifically, the inventor investigated, as the evaluation 1, the degree of change (percentage change) in the NOₓ output before and after the implementation of the aforementioned durability test for the examples 1 to 4, using the following model gas Mg1. That is, the inventor measured, for the examples 1 to 4, the NOₓ output when the model gas Mg1 with an NOₓ concentration of 1500 ppm and an O₂ concentration of 0% was caused to flow before and after the implementation of the durability test, and investigated the degree of change. As a result, the percentage changes were −6% in the example 1, −8% in the example 2, −10% in the example 3, and −5% in the example 4, as shown in “EVALUATION 1” in Table 1.

The inventor also investigated, as the evaluation 2, the degree of change (percentage change) in the NOₓ output before and after the implementation of the aforementioned durability test for the examples 1 to 4, using the following model gas Mg2. That is, the inventor measured, for the examples 1 to 4, the NOₓ output when the model gas Mg2 with an NOₓ concentration of 500 ppm and an O₂ concentration of 18% was caused to flow before and after the implementation of the durability test, and investigated the degree of change. As a result, the percentage changes were −7% in the example 1, −9% in the example 2, −10% in the example 3, and −5% in the example 4, as shown in “EVALUATION 2” in Table 1.

Further, the inventor investigated, as the evaluation 3, the percentage change in the offset value before and after the implementation of the aforementioned durability test for the examples 1 to 4, using the following model gas Mg3. That is, the inventor measured, for the examples 1 to 4, the offset value when the model gas Mg3 with an NOₓ concentration of 0 ppm, an O₂ concentration of 0%, and an H₂O concentration of 3% was caused to flow before and after the implementation of the durability test, and investigated the percentage change. As a result, the percentage changes were +4 ppm in the example 1, +5 ppm in the example 2, +7 ppm in the example 3, and +3 ppm in the example 4, as shown in “EVALUATION 3” in Table 1.

As shown in Evaluation 1, all of the gas sensors according to the examples 1 to 4 can keep the percentage change in the NOₓ output at 10% or less in an environment where the NOₓ concentration is 1500 ppm and the O₂ concentration is 0%, even after having been used for 2000 hours. Accordingly, it was confirmed that the gas sensor according to the present invention can realize highly accurate concentration measurement in an environment where the concentration of a specific gas (e.g., NOₓ) in the measurement target gas (e.g., exhaust gas) was high, even after having been used for a predetermined length of time.

As shown in Evaluation 2, all of the gas sensors according to the examples 1 to 4 can keep the percentage change in the NOₓ output at 10% or less in an environment where the NOₓ concentration is 500 ppm and the O₂ concentration is 18%, even after having been used for 2000 hours. Accordingly, it was confirmed that the gas sensor according to the present invention can realize highly accurate concentration measurement in an environment where the concentration of the specific gas in the measurement target gas was low (the concentration of a gas component other than the specific gas, such as O₂, was high), even after having been used for a predetermined length of time.

Specifically, as indicated as the evaluations 1 and 2, all of the gas sensors according to the examples 1 to 4 can keep the percentage change in the NOₓ output at 10% or less in both environments where the concentration of the specific gas in the measurement target gas is high and where the concentration of the specific gas is low. Accordingly, it was confirmed that the gas sensor according to the present invention can realize highly accurate concentration measurement in both environments where the concentration of the specific gas in the measurement target gas was high and where the concentration was low.

As shown in Evaluation 3, all of the gas sensors according to the examples 1 to 4 can keep the offset change amount at +7 ppm or less in an environment where the NOₓ concentration is 0 ppm, the O₂ concentration is 0%, and the H₂O concentration is 3%, even after having been used for 2000 hours. Accordingly, it was confirmed that the gas sensor according to the present invention can suppress the change in the offset value and measure the concentration of the specific gas in the measurement target gas with high accuracy. Note that the evaluation 3 is an evaluation of the amount of change in the offset value in the examples 1 to 4 before and after the implementation of the aforementioned durability test. Therefore, a method of correcting a fixed value every certain period of time, i.e., time correction can be used. Accordingly, if the amount of change in the offset value before and after the implementation of the aforementioned durability test is in the range from −10% to +10%, the aforementioned time correction can be effectively used, and it can be evaluated that a change in the offset value has been suppressed.

### LIST OF REFERENCE NUMERALS

S, S1, S2 Gas sensor
100 Gas sensor element (sensor element)
111 Determination unit
112 Temperature setting unit
1 First substrate layer (solid electrolyte layer)
2 Second substrate layer (solid electrolyte layer)
3 Third substrate layer (solid electrolyte layer)
4 First solid electrolyte layer (solid electrolyte layer)
5 Spacer layer (solid electrolyte layer)
6 Second solid electrolyte layer (solid electrolyte layer)
7 Measurement target gas flow section (internal space)
44 Measurement electrode
23 Outer pump electrode
41 Measurement pump cell
70 Heater (heater unit)
22 Inner pump electrode
51 Auxiliary pump electrode (inner pump electrode)
21 Main pump cell (adjustment pump cell)
50 Auxiliary pump cell (adjustment pump cell)

What is claimed is:

1. A gas sensor comprising:
a sensor element formed by stacking a plurality of solid electrolyte layers having oxygen ion conductivity, the sensor element including:
an internal cavity into which a measurement target gas is to be introduced;
a measurement pump cell being an electrochemical pump cell including: a measurement electrode located in the internal cavity; an outer pump electrode located in a region different from the internal cavity; and a solid electrolyte layer, of the plurality of solid electrolyte layers, that is present between the measurement electrode and the outer pump electrode; and
a heater unit embedded in the sensor element and configured to heat the sensor element to a specific temperature;
a determination unit configured to determine, based on output of the measurement pump cell, whether a concentration of a predetermined gas component in the measurement target gas is higher or lower than a predetermined concentration; and
a temperature setting unit configured to, if the determination unit determines that the concentration is lower, set the specific temperature to be lower than the specific temperature set if the determination unit determines that the concentration is higher.

2. The gas sensor according to claim 1,
wherein a slope of cell resistance of the measurement pump cell with respect to input power to the heater unit is 200 [ohm/W] or more.

3. The gas sensor according to claim 1,
wherein a slope of cell resistance of the measurement pump cell with respect to input power to the heater unit is 5000 [ohm/W] or less.

4. The gas sensor according to claim 1,
wherein the sensor element further includes at least one adjustment pump cell, which is an electrochemical pump cell including:
an inner pump electrode facing the internal cavity;
the outer pump electrode, or a third electrode in contact with a solid electrolyte layer, of the plurality of solid electrolyte layers, and exposed to an external space; and
a solid electrolyte layer, of the plurality of solid electrolyte layers, that is present between the inner pump electrode and the outer pump electrode or the third electrode,
the measurement target gas from which oxygen contained therein has been pumped in the adjustment pump cell is introduced into the measurement pump cell, and
a slope of cell resistance of the measurement pump cell with respect to input power to the heater unit is larger than a slope of cell resistance of the adjustment pump cell with respect to the input power to the heater unit.

5. The gas sensor according to claim 4,
wherein, if the determination unit determines that the concentration is lower, a value of the cell resistance of the measurement pump cell is controlled so as to be a predetermined first value, and
if the determination unit determines that the concentration is higher, the value of the cell resistance of the measurement pump cell is controlled so as to be a predetermined second value different from the first value.

6. The gas sensor according to claim 4,
wherein the slope of the cell resistance of the measurement pump cell with respect to the input power to the heater unit is 10 to 1000 times the slope of the cell resistance of the adjustment pump cell with respect to the input power to the heater unit.

7. A method for controlling a gas sensor including a sensor element formed by stacking a plurality of solid electrolyte layers having oxygen ion conductivity, the sensor element including:
an internal cavity into which a measurement target gas is to be introduced;
a measurement pump cell being an electrochemical pump cell including: a measurement electrode located in the internal cavity; an outer pump electrode located in a region different from the internal cavity; and a solid electrolyte layer, of the plurality of solid electrolyte layers, that is present between the measurement electrode and the outer pump electrode; and
a heater unit embedded in the sensor element and configured to heat the sensor element to a specific temperature,
the method comprising:
a determination step of determining, based on output of the measurement pump cell, whether a concentration of a predetermined gas component in the measurement target gas is higher or lower than a predetermined concentration; and
a temperature setting step of, if it is determined in the determination step that the concentration is lower, making the specific temperature lower than the specific temperature set if it is determined that the concentration is higher.
